(19) United States
(12) Patent Application Publication
(10) Pub. No.: US 2026/0108314 A1
(43) Pub. Date: Apr. 23, 2026

(54) BIOPRINTING ROBOTIC SYSTEM, DEVICE, AND METHOD

(71) Applicants: Board of Regents, The University of Texas System, Austin, TX (US); Terasaki Institute for Biomedical Innovation, Los Angeles, CA (US)

(72) Inventors: Farshid Alambeigi, Austin, TX (US); Samad Ahadian, Los Angeles, CA (US); Ali Khademhosseini, Los Angeles, CA (US); Johnson Vithrikunnil John, Torrance, CA (US)

(21) Appl. No.: 19/117,672

(22) PCT Filed: Oct. 4, 2023
(86) PCT No.: PCT/US2023/075898

§ 371 Date: Apr. 2, 2025

Related U.S. Application Data
(60) Provisional application No. 63/378,281, filed on Oct. 4, 2022.

(51) Int. Cl. A61B 34/32; A61B 34/10; A61B 34/20; A61B 34/35; A61B 90/00; A61B 90/30; A61B 90/50; B33Y 10/00; B33Y 30/00; B33Y 50/02

(52) U.S. Cl. CPC A61B 34/32; A61B 34/20; A61B 34/35; A61B 90/30; A61B 90/36; A61B 90/50; B33Y 10/00; B33Y 30/00; B33Y 50/02; A61B 2034/107; A61B 2034/2055; A61B 2090/365; A61B 2090/502

(57) ABSTRACT

A bioprinting robotic system comprises a robotic manipulator, a bioprinting instrument configured as an end effector of the robotic manipulator further configured to print bioink, a head mounted display (HMD), an optical tracking system, and a computing system communicatively connected to the robotic manipulator, bioprinting instrument, HMD and optical tracking system. A bioprinting instrument comprises a housing, a distance measurement sensor, a light source, 3D point cloud camera, and a co-axial nozzle positioned on a one degree of freedom (DoF) linear height control mechanism. A bioprinting method comprises providing the bioprinting robotic system above, obtaining a high-resolution volumetric geometry of an injury via the 3D point cloud camera, designing a desired 3D printing geometry and corresponding printing trajectories, calibrating and registering the bioprinting robotic system, displaying on the HMD the desired printing trajectory, and printing along the desired printing trajectory via a surgeon manipulating the bioprinting instrument.

### CROSS-REFERENCE TO RELATED APPLICATIONS

This application claims priority to U.S. provisional application No. 63/378,281 filed on Oct. 4, 2022, incorporated herein by reference in its entirety.

### STATEMENT REGARDING FEDERALLY SPONSORED RESEARCH OR DEVELOPMENT

This invention was made with government support under DP2 AR082471 awarded by the National Institutes of Health. The government has certain rights in the invention.

### BACKGROUND OF THE INVENTION

A total of 65.8 million Americans suffer from musculoskeletal injuries annually, with treatment costs exceeding 176 billion dollars. Musculoskeletal conditions comprise the second-highest global volume of years lived with disability. According to the CDC, it is estimated that these injuries result in an additional 326 billion dollars annually in lost productivity. Severe musculoskeletal injuries can lead to volumetric muscle loss (VML), where extensive musculoskeletal damage and tissue loss result in permanent loss of function. VML injuries can result from sports injuries, surgical resection, and traumatic events, such as car accidents and combat injury. In small-scale injuries or strains, muscle is capable of endogenous regeneration and complete functional restoration. However, this ability is abated in VML, where the native biophysical and biochemical signaling cues are no longer present to facilitate tissue regeneration.

Free functional muscle transfer (FFMT) is currently the only surgical treatment procedure for VML injuries, which has shown limited success in improving muscle function. FFMT is a complicated and time-consuming surgical procedure demanding the expertise of skilled orthopedics and microvascular surgeons, which limits its widespread application. Moreover, FFMT has complications such as infection, graft failure, and donor site morbidity due to tissue necrosis leading to a need for revision surgery or amputation of the affected limb. Prosthetic bracing is another treatment paradigm for patients with VML. Although the complications of surgical procedures are avoided, prosthetics must be custom-made for each patient, which is costly. Braces also fail to restore the native structure and function of the muscle and do not work for all types of VML. Due to the lack of a definitive treatment, VML often leads to permanent disability and pain, despite multiple interventions.

To overcome the mentioned limitations of current clinical treatments for VML injuries, many researchers around the world have been engaged in the development of nano- and micro-fabrication technologies to engineer native-like muscle tissues for muscle regeneration. Typically, in these tissue engineering procedures, cells from patients or donors are manually seeded into porous scaffolds to form artificial muscle tissues for implantation. However, randomly organized cells have poorly contributed to the regeneration of muscles. More specifically, the alignment and maturation of muscular cells are critical factors for engineering functional muscle tissues [See Science; 2017 (6); 356 (6342), incorporated herein by reference in its entirety]. Although many tissue engineering strategies have been utilized to fabricate muscle tissues and their corresponding extra cellular matrix, the capability for development of a three dimensional (3D) spatial cell organization that favors cells maturation and differentiation cascade is still lacking [See Tissue Eng Part B Rev; 2014 (10); 20 (5), incorporated herein by reference in its entirety].

In vitro 3D bioprinting is an innovative technology widely studied to construct tissue-like 3D structures, aiming to overcome the mentioned limitations by precise deposition of small units of living cells, biomaterials, and bioactive factors, to engineer skeletal muscle tissues. While in vitro bioprinting strategies can offer fabrication of complex scaffolds, they have several drawbacks as follows: bioprinted muscle constructs suffer from a prolonged culturing period in bioreactors demanding functionality enhancement prior to implantation in the body; in vitro 3D printed hydrogel scaffolds, whether injected, sutured, or placed into the wound, often fail to adhere to the remnant muscle and, therefore, do not provide adequate support for enduring mechanical loading; and most currently available stationary bioprinters are unable to print scaffolds on curved surfaces [See Acta Biomater; 2020 (1); 101:14-25, incorporated herein by reference in its entirety]. An emerging strategy to overcome these barriers is to directly bioprint a scaffold into the defect sites in situ.

To perform in situ fabrication of tissues, manual handheld in situ bioprinting systems have recently been proposed to directly deliver bioinks into the injury site to repair skin, cartilage, and muscle defects. Despite the benefits of using these devices and obtained promising results, these systems suffer from various limitations including poor manual control of the handheld bioprinter directly affected by the user's muscle fatigue and hand tremor during the printing procedure. These handheld devices are typically bulky and non-ergonomic making their control difficult particularly in long surgeries; heavily relying on the user's expertise and experience on using the bioprinting device and controlling the printing procedure, and lack of displaying the desired/planned printing trajectory and providing feedback to the user during the printing procedure (i.e., open-loop printing). These shortcomings lead to a long and hard-to-use printing procedure for large 3D injuries, non-uniform deposition, inaccurate stacking of multi-layer constructs, and most importantly, the lack of precision in the reconstruction of complex 3D curved and complex anatomical shapes leading to random and unorganized deposition and poor maturation of cells contributing to regeneration of muscles. Due to these issues, handheld bioprinters can typically be used to print simple 2D structures (e.g., skin repair) or filling gaps for cartilage and bone repair in which minimal precision is required. However, VML injuries typically have irregular large 3D geometries that demand precise bioprinting procedures to not only facilitate the complete functional restoration but also consider the cosmetic aspect of the muscle after surgery [See Acta Biomater; 2020 (1); 101:14-25, incorporated herein by reference in its entirety].

Autonomous bioprinting robotic systems have also been recently proposed for in situ bioprinting of human tissues. These systems, however, are bulky and do not provide enough degree-of-freedom (DoF) (mostly limited to X-Y-Z motions) to print complex 3D structures on different anatomies; do not consider a realistic surgical setting and workflow in the operating room. One example system covers the whole patient's body and does not consider a patient's inadvertent movement during the surgery to automatically adjust robotic pre-planned printing motions, thus endangering a patient's safety and reducing the printing accuracy; do not involve the surgeon during the bioprinting procedure to take advantage of a surgeon's skills and intuition and enhance the safety and success of procedure. Due to these issues, existing robotic bioprinters can typically be used for limited applications (e.g., skin repair) with 2D and small-size injuries. However, VML injuries typically have irregular large 3D geometries that demand precise bioprinting procedures to ensure complete functional and cosmetic restoration of the muscle after surgery.

Overall, a review of the literature (e.g., [See Acta Biomater; 2020 (1); 101:14-25, incorporated herein by reference in its entirety]) reveals that, so far, most of the researchers' efforts have been focused on the tissue engineering aspect of this important medical problem. Nevertheless, addressing this complex multidisciplinary challenge demands various expertise and close collaborations between researchers from different fields (i.e., the tissue engineering, surgical robotics, and clinical community) to develop advanced complementary bioinks and clinically-relevant in situ robotic delivery systems to precisely fabricate 3D constructs in the injury site for simultaneous functional and cosmetic restoration of muscles.

Thus, there is a need in the art for improved bioprinting robotic systems, devices, and methods.

### SUMMARY OF THE INVENTION

Some embodiments of the invention disclosed herein are set forth below, and any combination of these embodiments (or portions thereof) may be made to define another embodiment.

In one aspect, a bioprinting instrument comprises a housing, a distance measurement sensor, a light source, a 3D point cloud camera, and a nozzle positioned on a one degree of freedom (DoF) linear height control mechanism.

In one aspect, a bioprinting instrument comprises a housing including first and second ends, a distance measurement sensor positioned on the housing proximate to the first end, a light source positioned on the housing proximate to the first end, a 3D point cloud camera positioned on the housing, and a nozzle positioned on a one degree of freedom (DoF) linear height control mechanism positioned on the housing proximate to the first end.

In one embodiment, the bioprinting instrument is configured to be held by a surgeon.

In one embodiment, the bioprinting instrument is configured for in situ deposition of bioink.

In one embodiment, the bioink comprises gelatin-methacryloyl (GelMA) incorporating sustained insulin-like growth factor-1 (IGF-1) and myoblast cells.

In one embodiment, the bioink comprises any suitable bio-compatible ink, natural biomaterials such as alginate, gelatin, collagen, fibrin, fibrinogen, gellan gum, silk, hyaluronic acid, dextran, agarose, chitosan, hydroxyapatite, decellularized matrix based bioinks, growth factor based bioinks, Matrigel, synthetic biomaterials such as PCL, PEG, Pluronic, HAMA-pHPMA-lac/PEG, PG-HA, PVP, cell aggregate or pellet based bioinks, commercially available bioinks such as Derma-matrix and Novogel, and composite bioinks or bioinks with bioactive molecules such as AuNPs, AgNPs, magnetic iron oxide particles, blood plasma, cryo bioink, ultrashort peptides, genetically engineered phage, and conductive bioink.

In one embodiment, the nozzle has a diameter of 50 μm to 1000 μm, and comprises a coaxial nozzle, a single mode nozzle, a multimode nozzle, a syringe, and/or a chaotic mixing nozzle.

In one embodiment, the light source comprises a visible/UV light source.

In one embodiment, the bioprinting instrument further includes an ultrasound source configured for bioink cross linking.

In one embodiment, the bioprinting instrument utilizes an extrusion pressure of 0.1 kPa to 500 kPa.

In another aspect a bioprinting robotic system comprises a robotic manipulator, the bioprinting instrument as described above connected to and configured as an end effector of the robotic manipulator, further configured to print bioink a head mounted display (HMD), an optical tracking system, and a computing system communicatively connected to the robotic manipulator, bioprinting instrument, HMD and optical tracking system.

In one embodiment, the computing system comprises a processor and a non-transitory computer-readable medium with instructions stored thereon, which when executed by a processor, perform steps comprising obtaining a high-resolution geometry of a volumetric muscle loss (VML) injury via the 3D point cloud camera, designing a desired 3D printing geometry and corresponding printing trajectories, calibrating and registering the bioprinting robotic system, and displaying on the HMD the desired printing trajectory.

In one embodiment, the robotic manipulator comprises a 6 or 7 degrees of freedom redundant robotic manipulator.

In one embodiment, the system further comprises rigid body markers utilized by the optical tracking system as reference points for calibration, registration, and real time tracking of the system components and the patient.

In one embodiment, the system is configured to operate in a semi-autonomous cooperative SIL-RBP mode with visual augmented guidance.

In one embodiment, the system is configured to operate in a semi-autonomous cooperative SIL-RBP mode with visual augmented guidance virtual fixture guidance mode.

In one embodiment, the robotic manipulator, the bioprinting instrument, the HMD, the optical tracking system and the computing system are communicatively connected via wired, wireless, or wired and wireless means.

In one embodiment, the system utilizes co-operative shared control and virtual fixture algorithms to ensure safety and precision during the bioprinting procedure.

In one embodiment, the system is configured to operate in an assistive mode in which a surgeon holds the robot or bioprinting instrument and the system corrects the motions of the surgeon.

In one embodiment, the system is configured to operate in a semi-autonomous mode in which a portion of the motions are controlled by a surgeon and the remaining portion of the motions is controlled by the robot.

In on embodiment, the robot is configured to correct the motions of the surgeon.

In one embodiment, the system is configured to operate in a tele bioprinting mode in which a surgeon remotely controls a remote robot via another user interface or a leader robotic system.

In one embodiment, the system is configured to provide haptic, audio or visual feedback to the surgeon.

In one embodiment, the system is configured to operate in an autonomous mode in which the entirety of the bioprinting can be controlled autonomously based on the determined printing trajectory.

In one embodiment, the system is configured to perform spatial or planar bioprinting.

In another aspect, a bioprinting method comprises providing the bioprinting robotic system as described above, obtaining a high-resolution volumetric geometry of an injury via the 3D point cloud camera, designing a desired 3D printing geometry and corresponding printing trajectories, calibrating and registering the bioprinting robotic system, displaying on the HMD the desired printing trajectory, and accepting input from a surgeon to print along the desired printing trajectory via a manipulation of the bioprinting instrument.

In one embodiment, the method further comprises utilizing an algorithm to guide and scale a surgeon's movement to provide precise micromanipulation.

In one embodiment, the method further comprises monitoring the procedure and inadvertent patient movement via the optical tracker and 3D point cloud camera to cancel or correct the surgeon's manipulation of the bioprinting instrument.

In one embodiment, the step of designing a desired 3D printing geometry and corresponding printing trajectories comprises considering the point cloud data, clinician's feedback, properties of the engineered bioink, and biomechanics of the anatomy.

In one embodiment, the step of calibrating and registering the bioprinting robotic system comprises utilizing the optical tracking system and software to calibrate and register the printing instrument, HMD, 3D point cloud camera, and the robotic manipulator.

In another aspect, a bioprinting system comprises a robotic manipulator, a bioprinting instrument connected to and configured as an end effector of the robotic manipulator, further configured to print bioink, a display, an optical tracking system, and a computing system communicatively connected to the robotic manipulator, bioprinting instrument, display and optical tracking system, comprising a processor and a non-transitory computer-readable medium with instructions stored thereon, which when executed by a processor, perform steps comprising obtaining a high-resolution geometry of a target site via a 3D point cloud camera, designing a desired 3D printing geometry and corresponding printing trajectories, calibrating and registering the bioprinting robotic system, and displaying on the desired printing trajectory.

In one embodiment, the computing system further performs the step comprising printing bioink at the target site via the robotic manipulator and the bioprinting instrument.

In one embodiment, the system is configured to perform spatial or planar bioprinting.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIG. 1 depicts an exemplary computing environment in which aspects of the invention may be practiced in accordance with some embodiments.

FIG. 2A depicts an exemplary semi-autonomous in situ surgeon-in-the-loop (SIL) robotic bioprinting (RBP) system in accordance with some embodiments.

FIG. 2B depicts an exemplary tele-bioprinting system in accordance with some embodiments.

FIG. 2C depicts exemplary interface devices which may be utilized to control the bioprinting system in accordance with some embodiments.

FIG. 3A depicts exemplary 3D bioprinting constructs developed in vitro by incorporating PLGA-IGF-1 microparticles. A) Live-dead fluorescence assay at day 3 post-bioprinting. Living cells were stained by calcein (green) and dead cells by propidium iodide (red). B-C) DAPI-actin fluorescence staining of bioprinted cells after 3 days of culture. Cells nuclei were stained by DAPI (blue) and actin filaments by phalloidin (green). D-F) Immunofluorescent and brightfield images of myotubes formation inside of hydrogels containing PLGAIGF-1 microparticles, after 7 days post-bioprinting.

FIGS. 3B-3C depict planar and nonplanar (spatial) bioprinting in accordance with some embodiments.

FIG. 4A depicts an exemplary bioprinting device utilizing a co-axial extruding nozzle for deposition of the bioink in accordance with some embodiments. It also includes a distance measurement sensor, a high-frequency linear DoF, a small HD camera, and a light source.

FIGS. 4B-C depict an exemplary chaotic mixing nozzle in accordance with some embodiments.

FIGS. 4D-F depict additional exemplary bioprinting instruments which can be utilized as end effectors of the robot of the system in accordance with some embodiments. Any suitable bioprinting instrument including handheld instruments that can be configured as an end effector can be utilized.

FIG. 5 depicts exemplary unknown (dashed lines) and known (solid lines) rigid body transformations between different components of the SIL-RBP system in accordance with some embodiments.

FIG. 6 depicts exemplary registration and calibration procedures performed in accordance with some embodiments. [see IEEE ASME Trans Mechatron; 2021; 26 (1): 369-380, incorporated herein by reference in its entirety; and IEEE Robot Autom Lett.; 2017; (7); 2 (3): 1625-1631, incorporated herein by reference in its entirety]

FIG. 7 depicts an exemplary multi-objective optimization-based control and “virtual fixture” algorithms in accordance with some embodiments [see IEEE ASME Trans Mechatron; 2021; 26 (1): 369380, incorporated herein by reference in its entirety; and IEEE Robot Autom Lett.; 2017; (7); 2 (3): 1625-1631, incorporated herein by reference in its entirety].

FIG. 8 depicts a surgical robotic framework for in situ bioprinting and quantitative characterization in accordance with some embodiments.

FIG. 9 depicts an overall system framework including a bioprinting tool integrated with a seven-DoF robotic manipulator to perform a bioprinting procedure, a 3D visual measurement system with 2D/3D computer vision algorithms to enable online measurement and reconstruction of the geometric parameters of the bioprinted constructs, and a quantitative evaluation module with novel assessment metrics to characterize and evaluate performance of the bioprinting process.

FIG. 10 depicts a process for quantitative characterization of filament thickness including where a normal map of the pre-printed site is acquired through preoperative site scanning, where the pixels/sub-pixels belonging to the hydrogels are masked in the color images and the 3D filament point cloud is accessible given the segmented 2D masks (3D segmentation), and where the actual sizes of the filaments can be quantified by point cloud processing.

FIG. 11 depicts a Conceptual illustration of determining the thickness direction \vec{vₜ}, where \vec{nₛ} denotes the normal vector of the surface and \vec{vₗ} represents the filament direction, which is defined by connected median points.

FIG. 12 is a table showing quantitative evaluation results.

FIG. 13 depicts a visualization of the segmentation boundaries with and without SPR. The red curve and arrow indicate the segmentation boundaries and the aliased regions, respectively.

FIGS. 14A-14C depict quantitative characterization of the overall global errors and uniformity errors for each case, where yellow region, red region, and gray region denote overall global error, overall uniformity error, and failure (discontinuous) cases, respectively.

FIGS. 15A-15I depict quantitative evaluations for several cases. For each case, the continuous global error and uniform error are shown as normalized heat maps aligned with the segmentation map (Seg). The measured errors for each successful case (without disconnections): (a) E_{g}=1.65 mm, Eᵤ=0.21 mm (b) E_{g}=0.73 mm, Eᵤ=0.07 mm (c) E_{g}=0.41 mm, Eᵤ=0.08 mm (g) E_{g}=0.23 mm, Eᵤ=0.01 mm; (h) E_{g}=0.26 mm, Eᵤ=0.01 mm; (i) E_{g}=0.28 mm, Eᵤ=0.18 mm.

### DETAILED DESCRIPTION OF THE INVENTION

It is to be understood that the figures and descriptions of the present invention have been simplified to illustrate elements that are relevant for a clearer comprehension of the present invention, while eliminating, for the purpose of clarity, many other elements found in systems and methods of bioprinting robotics. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Ranges: throughout this disclosure, various aspects of the invention can be presented in a range format. It should be understood that the description in range format is merely for convenience and brevity and should not be construed as an inflexible limitation on the scope of the invention. Where appropriate, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Referring now in detail to the drawings, in which like reference numerals indicate like parts or elements throughout the several views, in various embodiments, presented herein are bioprinting robotic systems, devices and methods.

In view of current abovementioned limitations and the tremendous potential of bridging the existing gap between computer-assisted robotic surgery, tissue engineering, and 3D bioprinting fields, a novel surgical robotic system and complementary computer-assisted algorithms were developed to perform an unprecedented semi-autonomous in situ robotic bioprinting of functional muscles. With the long-range goal of treating various types of large-scale 3D VML injuries in animal models, this robotic system can utilize complementary tissue engineered bioinks. This robotic bioprinting system is the first-in-the-world in situ bioprinting system with real-time feedback on the precision of the 3D bioprinted constructs as well as the surgeon's and patient's motions to enable maturation of the in situ bioprinted tissue constructs by directly using human/animal body as an effective bioreactor, ensure safety and precision of the bioprinting procedure, and more importantly enable simultaneous functional and cosmetic muscle restoration.

### Computing Environment

In some aspects of the present invention, software executing the instructions provided herein may be stored on a non-transitory computer-readable medium, wherein the software performs some or all of the steps of the present invention when executed on a processor.

Aspects of the invention relate to algorithms executed in computer software.

Though certain embodiments may be described as written in particular programming languages, or executed on particular operating systems or computing platforms, it is understood that the system and method of the present invention is not limited to any particular computing language, platform, or combination thereof. Software executing the algorithms described herein may be written in any programming language known in the art, compiled or interpreted, including but not limited to C, C++, C#, Objective-C, Java, JavaScript, MATLAB, Python, PHP, Perl, Ruby, or Visual Basic. It is further understood that elements of the present invention may be executed on any acceptable computing platform, including but not limited to a server, a cloud instance, a workstation, a thin client, a mobile device, an embedded microcontroller, a television, or any other suitable computing device known in the art.

Parts of this invention are described as software running on a computing device. Though software described herein may be disclosed as operating on one particular computing device (e.g. a dedicated server or a workstation), it is understood in the art that software is intrinsically portable and that most software running on a dedicated server may also be run, for the purposes of the present invention, on any of a wide range of devices including desktop or mobile devices, laptops, tablets, smartphones, watches, wearable electronics or other wireless digital/cellular phones, televisions, cloud instances, embedded microcontrollers, thin client devices, or any other suitable computing device known in the art.

Similarly, parts of this invention are described as communicating over a variety of wireless or wired computer networks. For the purposes of this invention, the words “network”, “networked”, and “networking” are understood to encompass wired Ethernet, fiber optic connections, wireless connections including any of the various 802.11 standards, cellular WAN infrastructures such as 3G, 4G/LTE, or 5G networks, Bluetooth®, Bluetooth® Low Energy (BLE) or Zigbee® communication links, or any other method by which one electronic device is capable of communicating with another. In some embodiments, elements of the networked portion of the invention may be implemented over a Virtual Private Network (VPN).

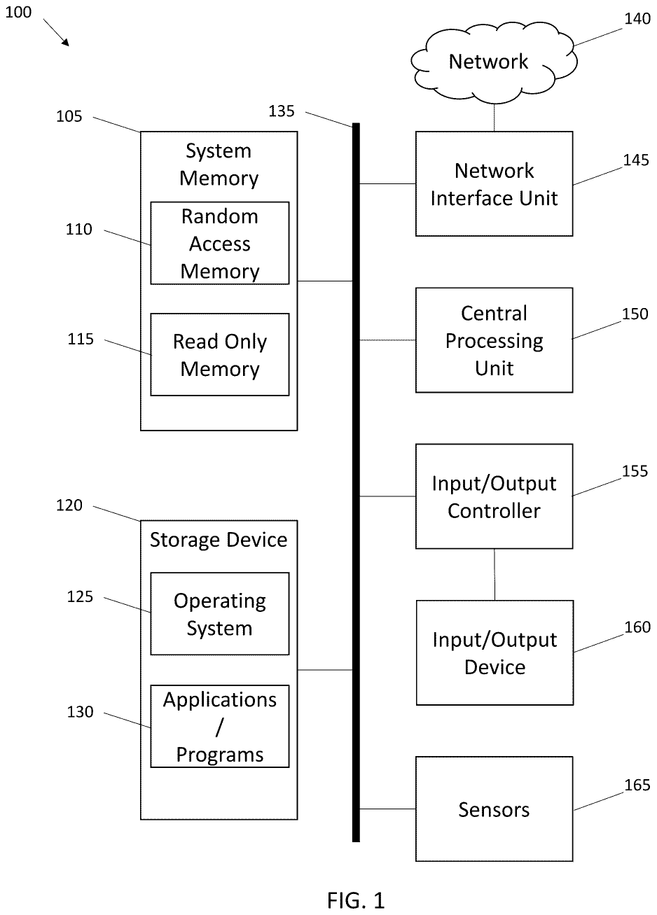

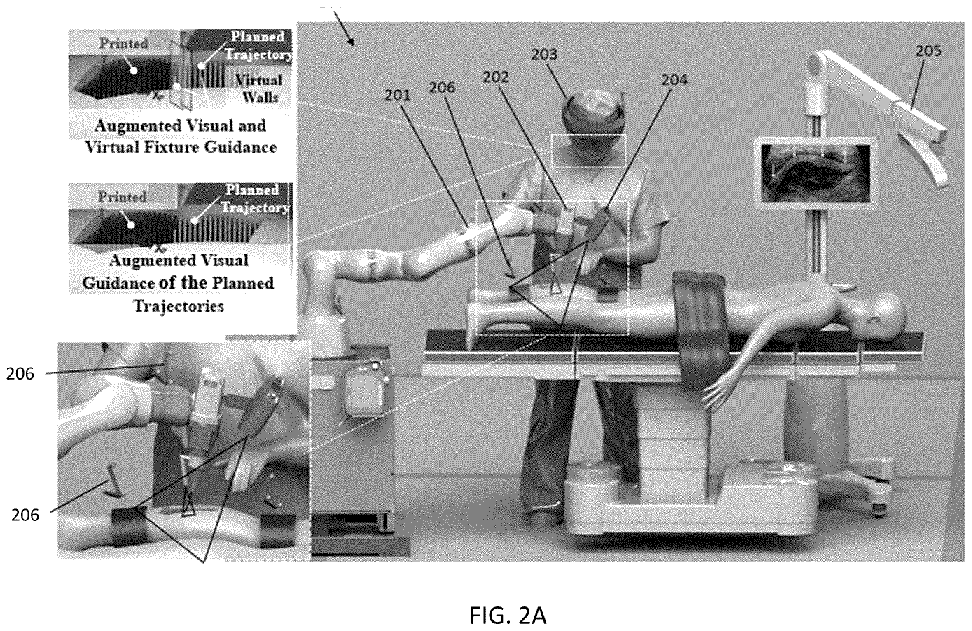

FIG. 1 and the following discussion are intended to provide a brief, general description of a suitable computing environment in which the invention may be implemented. While the invention is described above in the general context of program modules that execute in conjunction with an application program that runs on an operating system on a computer, those skilled in the art will recognize that the invention may also be implemented in combination with other program modules.

Generally, program modules include routines, programs, components, data structures, and other types of structures that perform particular tasks or implement particular abstract data types. Moreover, those skilled in the art will appreciate that the invention may be practiced with other computer system configurations, including hand-held devices, multiprocessor systems, microprocessor-based or programmable consumer electronics, minicomputers, mainframe computers, and the like. The invention may also be practiced in distributed computing environments where tasks are performed by remote processing devices that are linked through a communications network. In a distributed computing environment, program modules may be located in both local and remote memory storage devices.

FIG. 1 depicts an illustrative computer architecture for a computer 100 for practicing the various embodiments of the invention. The computer architecture shown in FIG. 1 illustrates a conventional personal computer, including a central processing unit 150 (“CPU”), a system memory 105, including a random-access memory 110 (“RAM”) and a read-only memory (“ROM”) 115, and a system bus 135 that couples the system memory 105 to the CPU 150. A basic input/output system containing the basic routines that help to transfer information between elements within the computer, such as during startup, is stored in the ROM 115. The computer 100 further includes a storage device 120 for storing an operating system 125, application/program 130, and data.

The storage device 120 is connected to the CPU 150 through a storage controller (not shown) connected to the bus 135. The storage device 120 and its associated computer-readable media, provide non-volatile storage for the computer 100. Although the description of computer-readable media contained herein refers to a storage device, such as a hard disk or CD-ROM drive, it should be appreciated by those skilled in the art that computer-readable media can be any available media that can be accessed by the computer 100.

By way of example, and not to be limiting, computer-readable media may comprise computer storage media. Computer storage media includes volatile and non-volatile, removable and non-removable media implemented in any method or technology for storage of information such as computer-readable instructions, data structures, program modules or other data. Computer storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

According to various embodiments of the invention, the computer 100 may operate in a networked environment using logical connections to remote computers through a network 140, such as TCP/IP network such as the Internet or an intranet. The computer 100 may connect to the network 140 through a network interface unit 145 connected to the bus 135. It should be appreciated that the network interface unit 145 may also be utilized to connect to other types of networks and remote computer systems.

The computer 100 may also include an input/output controller 155 for receiving and processing input from a number of input/output devices 160, including a keyboard, a mouse, a touchscreen, a camera, a microphone, a controller, a joystick, or other type of input device. Similarly, the input/output controller 155 may provide output to a display screen, a printer, a speaker, or other type of output device. The computer 100 can connect to the input/output device 160 via a wired connection including, but not limited to, fiber optic, ethernet, or copper wire or wireless means including, but not limited to, Bluetooth, Near-Field Communication (NFC), infrared, or other suitable wired or wireless connections.

As mentioned briefly above, a number of program modules and data files may be stored in the storage device 120 and RAM 110 of the computer 100, including an operating system 125 suitable for controlling the operation of a networked computer. The storage device 120 and RAM 110 may also store one or more applications/programs 130. In particular, the storage device 120 and RAM 110 may store an application/program 130 for providing a variety of functionalities to a user. For instance, the application/program 130 may comprise many types of programs such as a word processing application, a spreadsheet application, a desktop publishing application, a database application, a gaming application, internet browsing application, electronic mail application, messaging application, and the like. According to an embodiment of the present invention, the application/program 130 comprises a multiple functionality software application for providing word processing functionality, slide presentation functionality, spreadsheet functionality, database functionality and the like.

The computer 100 in some embodiments can include a variety of sensors 165 for monitoring the environment surrounding and the environment internal to the computer 100. These sensors 165 can include a Global Positioning System (GPS) sensor, a photosensitive sensor, a gyroscope, a magnetometer, thermometer, a proximity sensor, an accelerometer, a microphone, biometric sensor, barometer, humidity sensor, radiation sensor, or any other suitable sensor.

### Bioprinting Robotic System

Disclosed herein is a surgical robotic system and complementary intuitive computer-assisted algorithms that enables semi-autonomous surgeon-in-the-loop (SIL), fully autonomous, and/or remote semi-autonomous SIL, in situ bioprinting of tissues and organs for simultaneous functional and cosmetic restoration of various types of injuries such as skeletal muscle injuries.

The disclosed SIL in situ robotic bioprinting system can collectively address the abovementioned limitations of the current state-of-the-art manual handheld and autonomous robotic bioprinting systems by reducing complexity, surgical time, and complications associated with current VML treatments, immediately delivering and in situ printing of appropriate bioinks to the target anatomy on patient's body and utilizing the human body as a natural bioreactor to induce tissue maturation and function in situ, providing real-time feedback on the 3D bioprinted constructs as well as the surgeon's and patient's motions to ensure precision of the bioprinting procedure for simultaneous functional and cosmetic restoration of the injured muscle and providing an intuitive, ergonomic, and safe SIL semi-autonomous procedure for the surgeon using a robotic system that includes a robotic bioprinting instrument and complementary computer-assisted algorithms. This robotic system holds the weight of the bioprinting device to avoid muscle fatigue and subsequent hand tremor and is able to scale and correct surgeon's motion to perform precise micro-manipulation. It is worth emphasizing that the disclosed robotic system takes advantage of the features of both abovementioned manual handheld and fully-autonomous bioprinting technologies while completely overcoming their limitations. The system enables in situ bioprinting of engineered tissue constructs and bioinks at the injury site to ensure both functional and cosmetic muscle restoration. The systems and methods disclosed herein can also be generalized for any suitable handle held or robotic bioprinting application or surgery addressing, for example, small scale injuries such as crack filling, large scale injuries such as VML and skin burns, bone fracture, and bone loss by utilizing bioink specific to said application or surgery.

### System Overview

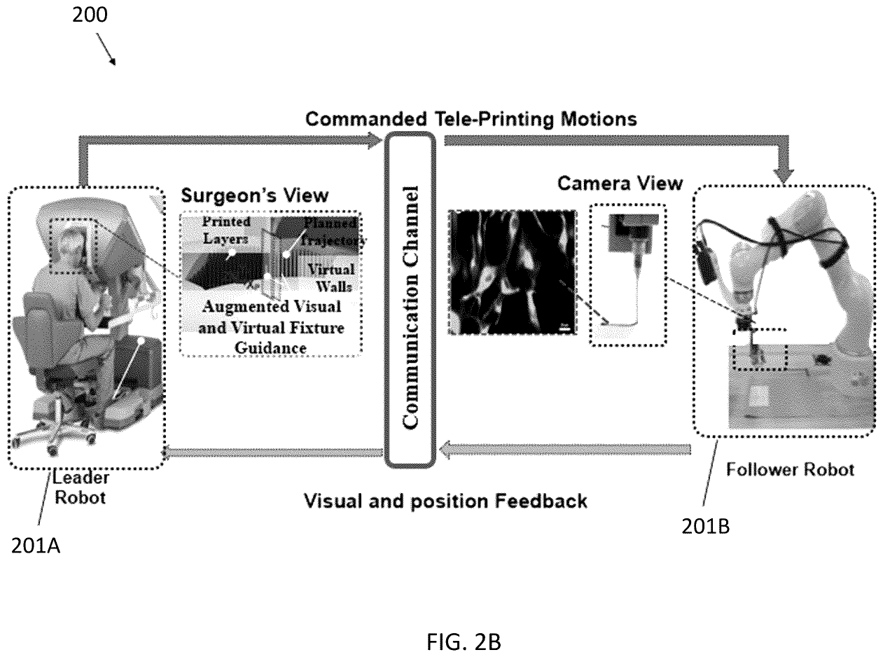

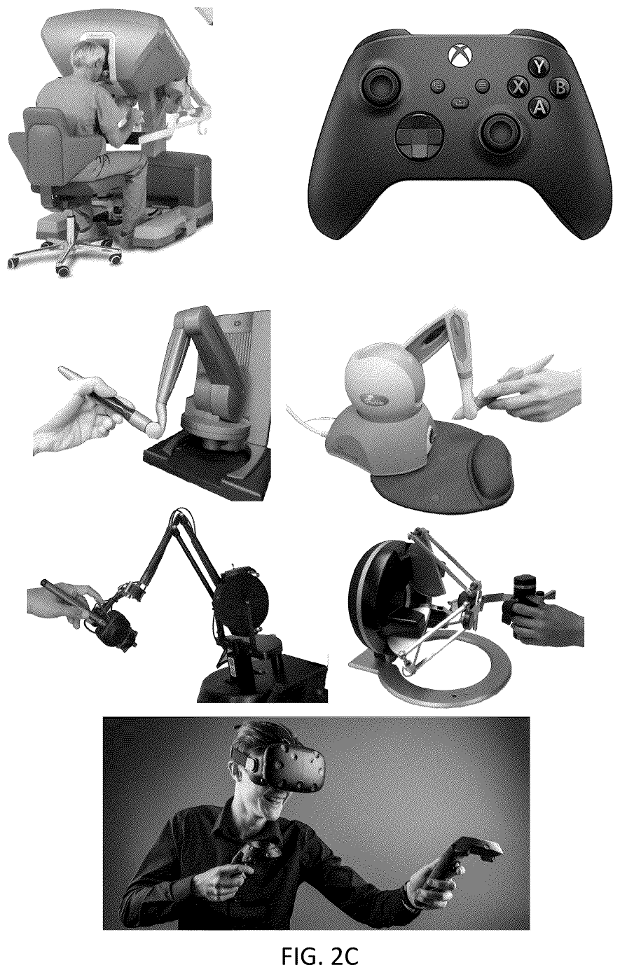

As shown in FIG. 2A, to perform a safe, intuitive, and precise semi-autonomous in situ SIL-RBP for simultaneous functional and cosmetic restoration of muscles with large VML injuries, skin burns, bone fracture, and/or bone loss, an RBP system 200 comprises the hardware, software, and SIL-RPB control components described below. In some embodiments, the system is configured to perform any suitable bioprinting from small scale printing such as crack filling, to large scale printing such as large area VML repair.

In some embodiments, as shown in FIG. 2A, the SIL-RBP system 200 comprises a robotic manipulator 201, such as a redundant robotic manipulator with 6-7 DoF (e.g., Kuka LBR, Kuka Inc. or Panda, Franka Emika GmbH). This robotic manipulator is integrated with a bioprinting instrument attached to its end effector. As shown, to directly control the bioprinting procedure, a surgeon holds the integrated bioprinting instrument 202. Then, the integrated robotic system measures the surgeon's hand motion/force commands and utilizes the computer-assisted robotic algorithms to filter and/or scale these motions to enable an intuitive, safe, and precise SIL-RBP of complex 3D constructs.

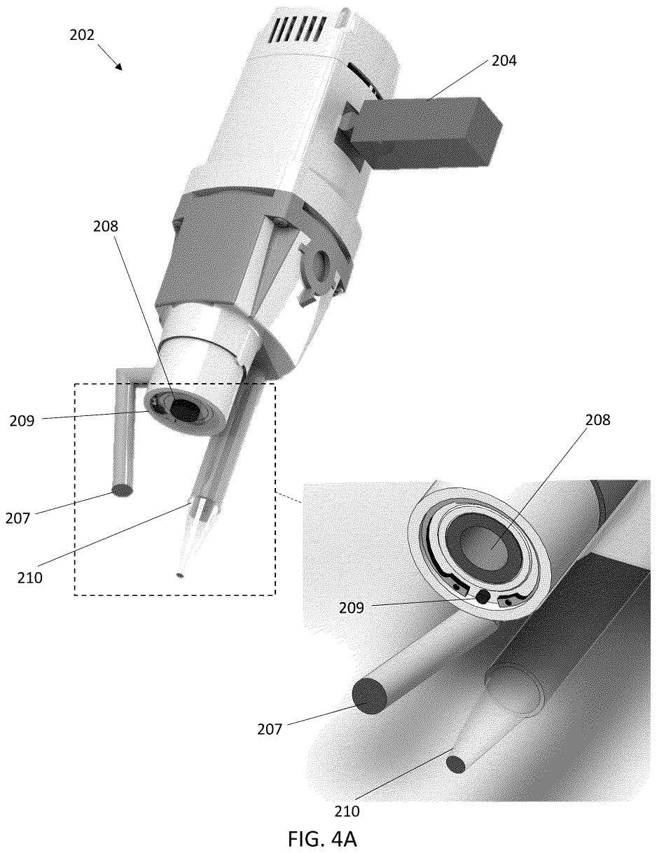

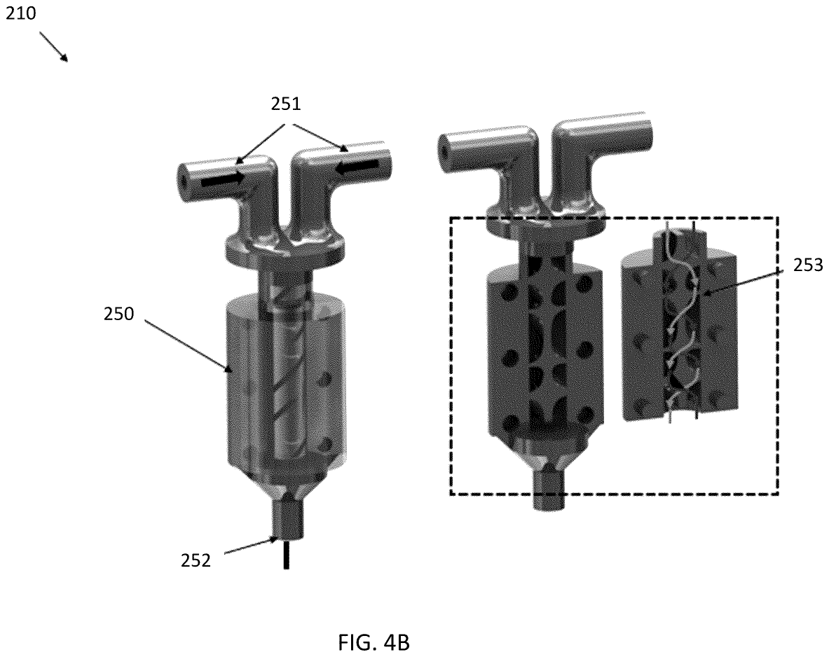

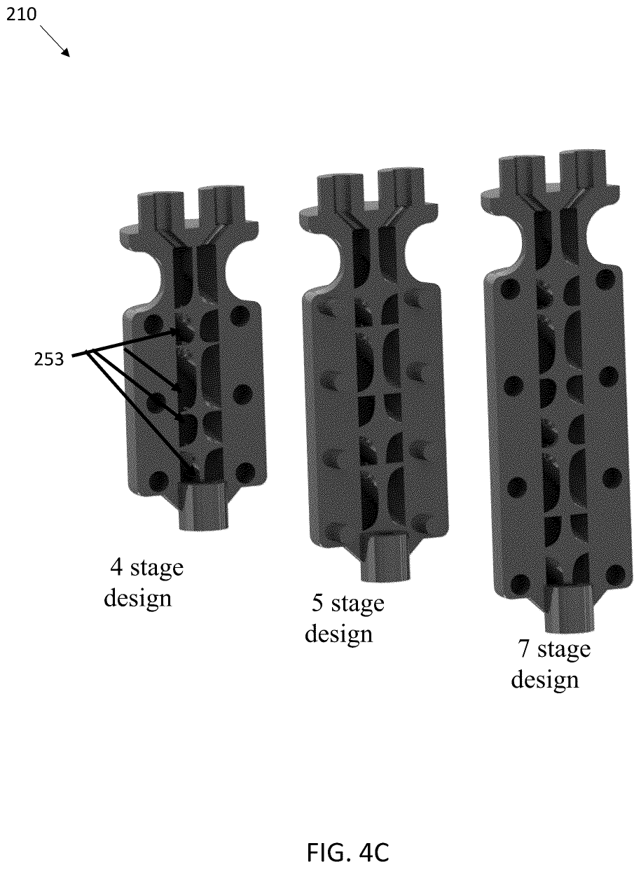

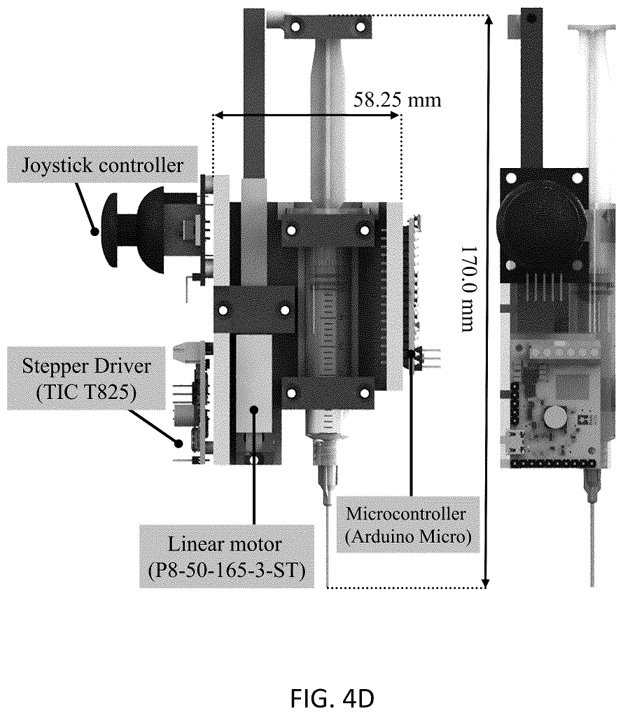

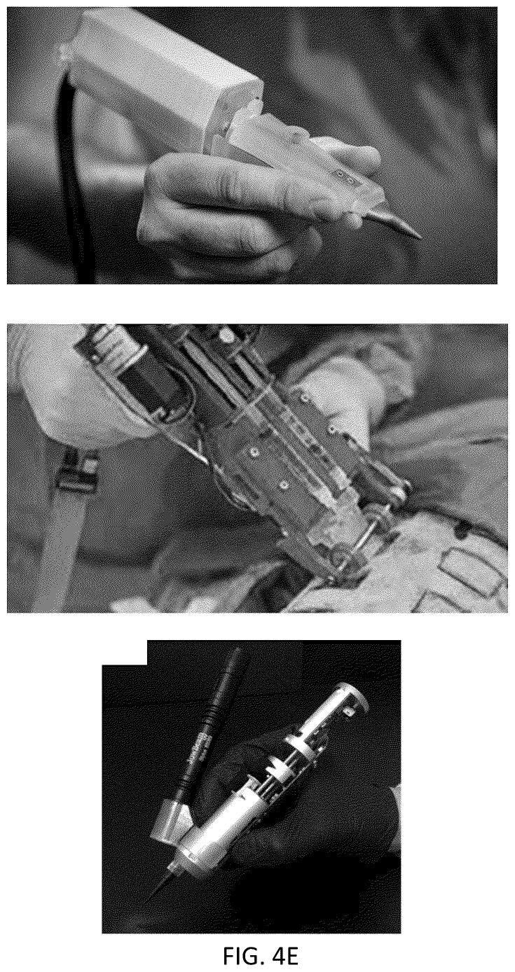

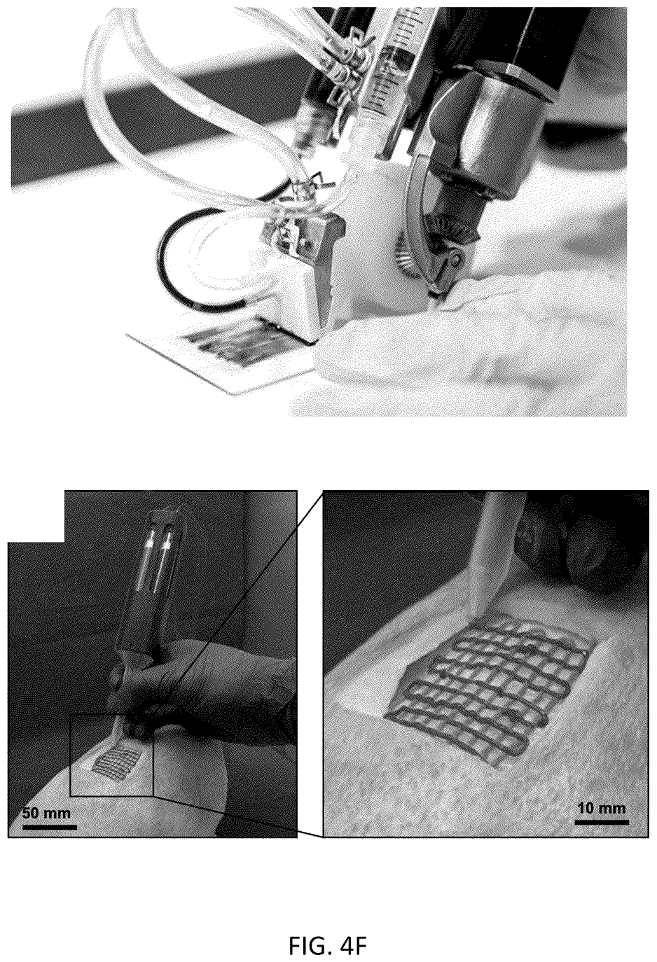

In some embodiments, as shown in FIGS. 2A and 4, system 200 comprises an ergonomic bioprinting instrument 202 configured for safe, uniform, and precise in situ deposition of the complementary engineered bioink. This instrument is equipped with appropriate sensors and is designed to serve as an independent module to be either integrated with a generic robotic manipulator 201 or controlled manually by the surgeon. In some embodiments, the bioprinting instrument 202 comprises an off the shelf bioprinter.

In some embodiments, the bioprinting instrument 202 comprises a housing, a distance measurement sensor 208, a light source 207, a 3D point cloud camera 204, and a nozzle 210 positioned on a one degree of freedom (DoF) linear height control mechanism. In some embodiments, the nozzle comprises a co-axial nozzle, a single mode nozzle, a multimode nozzle, a syringe, and/or a chaotic mixing nozzle. In some embodiments, the bioprinting instrument 202 is configured to be held by a surgeon. In some embodiments, the bioprinting instrument 202 is configured for in situ deposition of bioink. In some embodiments, the bioink comprises gelatin-methacryloyl (GelMA) incorporating sustained insulin-like growth factor-1 (IGF-1) and myoblast cells. In some embodiments, the nozzle 210 has a diameter of 250 μm to 500 μm. In some embodiments, the light source 207 comprises a visible/UV light source. In some embodiments, the bioprinting instrument 202 utilizes an extrusion pressure of 0.1 kPa to 500 kPa.

In some embodiments, the bioprinting instrument further includes an ultrasound source configured for bioink cross linking.

In some embodiments, the bioink comprises any suitable bio-compatible ink, natural biomaterials such as alginate, gelatin, collagen, fibrin, fibrinogen, gellan gum, silk, hyaluronic acid, dextran, agarose, chitosan, hydroxyapatite, decellularized matrix based bioinks, growth factor based bioinks, Matrigel, synthetic biomaterials such as PCL, PEG, Pluronic, HAMA-pHPMA-lac/PEG, PG-HA, PVP, cell aggregate or pellet based bioinks, commercially available bioinks such as Derma-matrix and Novogel, and composite bioinks or bioinks with bioactive molecules such as AuNPs, AgNPs, magnetic iron oxide particle, blood plasma, cryo bioink, ultrashort peptides, genetically engineered phage, and conductive bioink.

In some embodiments, a high-resolution 3D point cloud camera 204 is included and utilized before the procedure to obtain the precise 3D geometry of the injury site. In some embodiments, the camera 204 comprises any suitable RGBD camera that can provide a high resolution point cloud of the target site. Using this point cloud data, a 3D printing construct is designed to mimic the lost muscle anatomy. During the procedure, the 3D point cloud camera 204 is used to provide real-time 3D point cloud measurements of the bioprinted constructs in order to ensure and validate precise execution of the planned geometry.

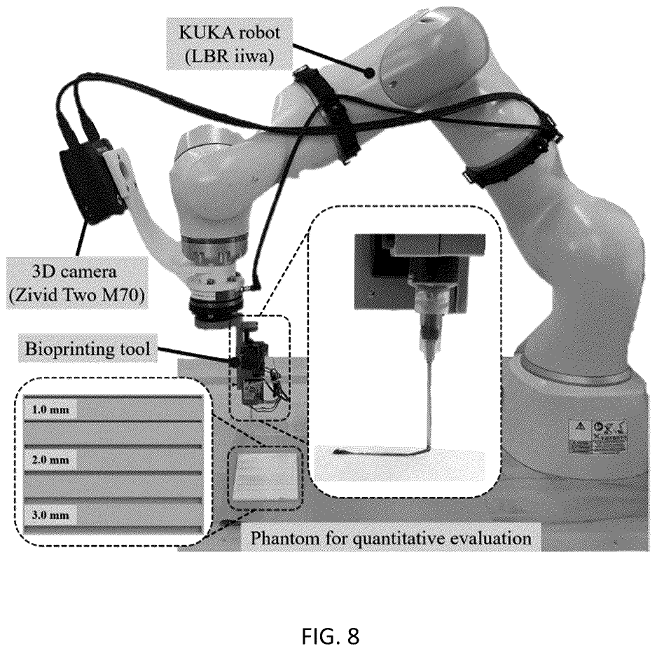

In some embodiments, computer vision techniques are utilized to quantitatively evaluate the quality of the print. Further details and examples of this are described below and shown in FIGS. 8-15.

In some embodiments, an optical see-through head mounted display (HMD) 203 is used to overlay and augment the desired printing trajectory and useful information during the printing procedure to the surgeon's direct view. Using this see-through HMD 203, a surgeon can easily walk around the patient while the displayed information is accordingly being updated in real-time based on his point of view and location.

In some embodiments, an optical tracking system 205 and complementary optical rigid bodies 206 are used for calibration and registration of the patient, the robot, HMD, and bioprinting instrument before the procedure, and to provide real-time tracking of the patient and robot 201 to ensure safety and precision of the procedure. It also can be used to update the augmented information on the HMD 203 while the surgeon is moving or changing his view during the procedure.

In some embodiments, a complementary bioink with the bioprinting instrument is used.

In some embodiments, to safely, intuitively, and precisely perform an in situ SIL-RBP procedure, various software components are implemented in the system. To perform bioprinting, desired printing geometry and trajectories need to be designed first and then augmented on the surgeon's HMD 203. This procedure demands calibration and registration of the 3D point cloud camera 204, HMD 203, optical tracker 205, robotic manipulator 201, and bioprinting instrument 202.

Based on the planned printing geometry and trajectories, appropriate kinematics and control algorithms are used to receive the motion and printing commands from the surgeon and then execute them using the integrated robotic system 200. Notably, in this control mode, surgeon, robotic manipulator 201, and computer co-operate and share control of the bioprinting instrument 202 to improve the safety and precision of the RBP in real-time. Safety of the procedure requires ensuring an appropriate distance from the anatomical surface, while printing precision demands a pre-defined constant distance between the instrument nozzle tip and the printing surface. Moreover, to follow a specific printing pattern/trajectory, the surgeon's motion needs to be guided and scaled to avoid hand tremors and the unintentional movements. To address these needs, appropriate “virtual fixture” algorithms were developed.

In some example embodiments, the system 200 is fully autonomous.

In some example embodiments, the system 200 comprises a robotic tele-bioprinting (RTB) system as shown for example in FIG. 2B. In some embodiments, the RTB system comprises a tele-surgical robotic platform comprising a leader (surgeon-side) robot 201A and follower (patient-side) robots 201B. The leader robot 201A directly interacts with the surgeon to obtain his/her motion commands using robotic manipulators and remotely control a bioprinting instrument 202. In some embodiments, the follower robot 201B receives the commands from the leader robot 201A to move the bioprinting instrument 202. FIG. 2B shows the leader 201A and follower 201B robots of an example da Vinci surgical robotic system (Intuitive Surgical Inc., CA). Of note, the leader 201A and follower 201B robots can be replaced by other appropriate robotic manipulators. In some embodiments, the bioprinting instrument 202 may be equipped with appropriate sensors and designed to serve as an independent robotic module to be integrated and intuitively controlled using a surgeon and a generic follower robotic arm (e.g., da Vinci patient side manipulators or Kula LBR manipulator).

To safely, intuitively, and precisely tele-operate the RTB system 200, the following software components are utilized. Printing trajectory planning, calibration, and registration algorithms are used to perform bioprinting at the print location, as printing trajectory needs to be designed first and then mapped and printed at the printing location based on patient anatomy. Co-operative tele-bioprinting algorithms may be based on the planned trajectory, and on appropriate kinematics and control algorithms which are developed to receive the motion and printing commands from the surgeon and execute the motions using the follower robotic system 201B. Notably, in this control mode the surgeon and computer share control of the robotic bioprinting nozzle 210 of the bioprinting instrument 202 to improve the safety and precision of the tele-bioprinting procedure. Intuitive co-operative tele-bioprinting algorithms may also be used to simultaneously ensure the safety, precision, and intuitiveness of the tele-bioprinting procedure. The robotic nozzle 210 needs to be remotely operated while precisely printing a pre-defined pattern at the print site. Safety of procedures requires ensuring a safe distance from the patient, while printing precision demands a pre-defined constant distance between the nozzle tip and print location on the patient. Moreover, to follow a specific printing pattern/trajectory, the surgeon's motion needs to be guided and scaled to avoid hand tremors and unintentional movements which can be mitigated via virtual fixtures as described herein.

### Procedure Workflow/Method

In some embodiments, to perform an SIL-RBP procedure, the below workflow method is utilized. First, a 3D point cloud camera 204 is used to obtain a high-resolution volumetric geometry of the injury. Then considering the point cloud data, clinician's feedback, properties of the engineered bioink, and biomechanics of the anatomy, a 2D or 3D desired printing geometry of the injury and the corresponding printing trajectories (e.g., path, resolution, speed, total time, and printing pattern) are designed in the CAD software. As shown in FIG. 2A, based on the location of injury, the integrated robotic system 200 is placed near the patient such that it provides adequate motion workspace for the surgeon during the printing procedure. Moreover, considering the posture of surgeon and location of the injury, optical tracker 205, rigid bodies 206, and the point cloud camera 204 are positioned to provide optimal field of views for tracking the robot 201, surgeon, and patient as well as monitoring the 3D printed structure in real-time, respectively.

Utilizing the optical tracking system 205, the software is used to calibrate and register the printing instrument 202, HMD 203, 3D point cloud camera 204, and the robotic manipulator 201. Moreover, this software was used to augment the planned printing trajectory on surgeon's HMD 203. The surgeon then holds the bioprinting instrument 202 in a comfortable and ergonomic standing or sitting posture. Next, desired printing trajectory and other guiding information are augmented to the HMD 203 and the surgeon directly controls the integrated robotic bioprinting system 200 to follow the displayed printing trajectory. During this process, the co-operative shared control and virtual fixture algorithms ensure safety and precision during the bioprinting procedure. In addition, using this algorithm, the surgeon's movement is guided and scaled to provide a precise micro manipulation. The surgeon has complete control through the process to start, pause, and restart the SILRBP procedure and deposition of the bioink. During the procedure, optical tracker 205 and 3D point cloud camera 204 also continuously monitor the whole procedure and particularly any inadvertent patient motions. Based on these feedbacks, shared control algorithms can cancel and/or correct the surgeon's motions to ensure a safe and precise bioprinting procedure.

In the example of the RTB system of FIG. 2B, to perform an RTB procedure, a surgeon may first use an appropriate imaging modality (e.g., MRI or CT) to determine the size, geometry, and location for the planned print. Using this information, the planning software calculates an optimal printing trajectory (e.g., path, speed, total time, and printing pattern) for the robotic procedure. Based on the target location of the print, the robotic nozzle may then be introduced to the patient's body using appropriate means. Next, the surgeon can prepare and clean the printing site if needed. Using the calibration and registration software, the planned printing trajectory is then mapped on the printing site anatomy and augmented on the surgeon's side monitor. The robotic nozzle and endoscope are also registered to the patient's anatomy. The robotic nozzle is then controlled and tele-operated by the surgeon. Co-operative shared control and virtual fixture algorithms ensure the safety and precision of the surgeon's motion during bioprinting. Moreover, the surgeon's movement is guided and scaled to provide a precise micro tele-bioprinting using engineered bioink. The surgeon has complete control through the process to start, pause, and restart the printing procedure.

### EXPERIMENTAL EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out exemplary embodiments of the present invention, and are not to be construed as limiting in any way the remainder of the disclosure.

To follow the described workflow, various challenges were addressed for the first time from hardware and software development to system integration and performing unprecedented performance evaluation studies with the developed bioinks.

### Development of Bioink

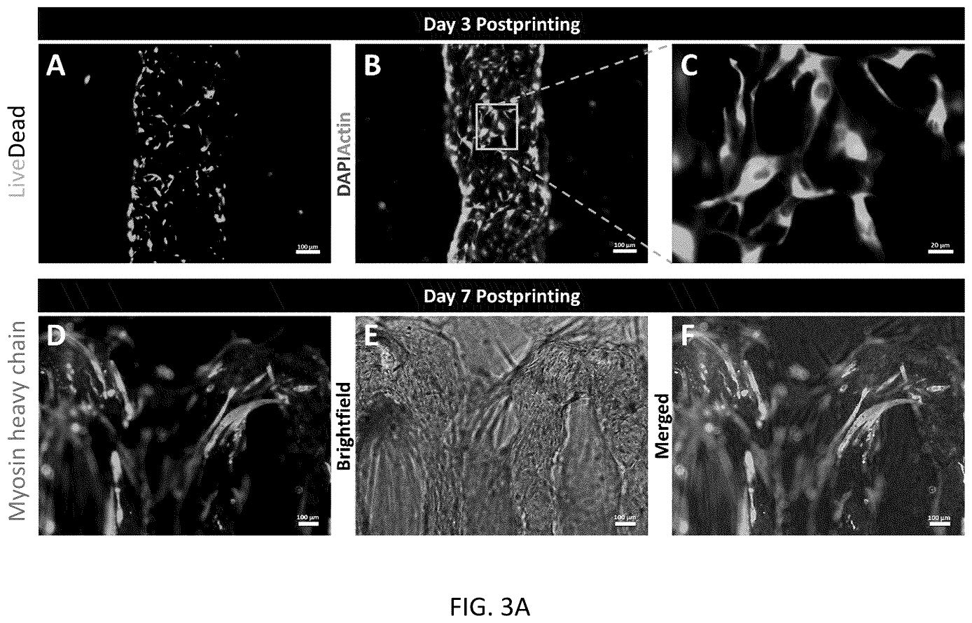

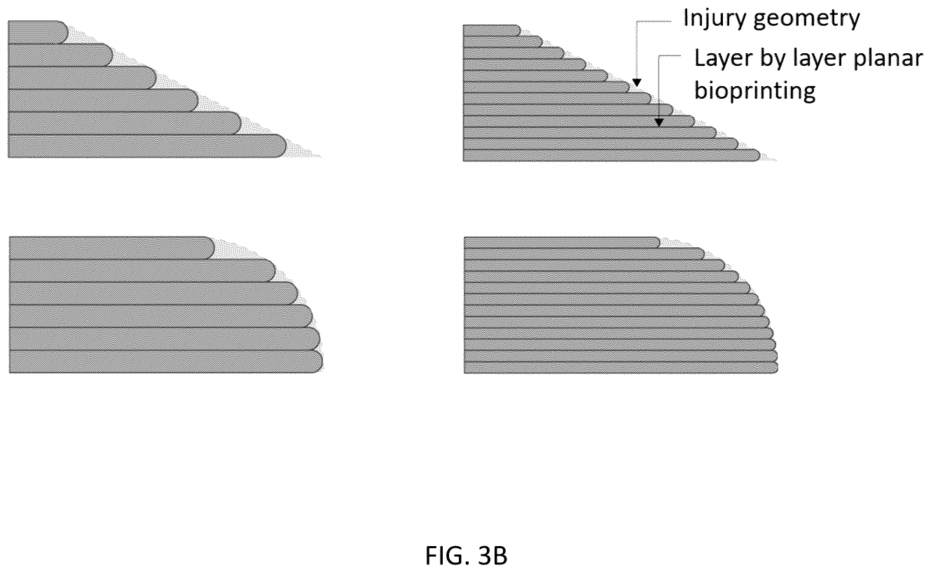

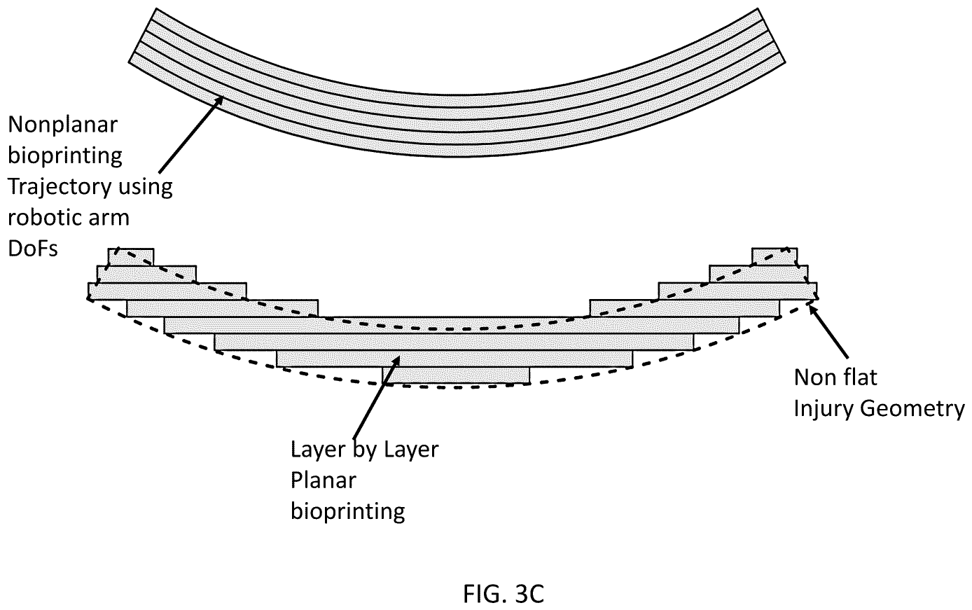

Example bioinks used for 3D bioprinting for in vitro fabrication of muscle tissues use gelatin-methacryloyl (GelMA) incorporating sustained insulin-like growth factor-1 (IGF-1) and myoblast cells (e.g., [see ACS Biomater. Sci. Eng. 2017, 3, 4; J Tissue Eng Regen Med; 2017 (2); 11(2), incorporated herein by reference in its entirety]. As shown in FIG. 3A, it has been demonstrated that culturing cells in the developed in vitro-engineered muscle bioink, releasing IGF-1 in a sustained manner, resulted in an enhancement of efficiency of obtained functional and mature myotubes. Interestingly, incorporation of IGF-1 in GelMA hydrogel assisted in promoting differentiation and alignment of myotubes. Furthermore, the developed myotubes have shown spontaneous contraction in the muscle constructs bioprinted with IGF-1 releasing bioink.

### Development of Bioprinting Instrument

To design a bioprinting instrument that was compatible with the engineered bioink and the disclosed robotic system 200, the following bioprinting and fabrication constraints were considered.

For successful bioprinting, rapid polymerization and adequate crosslinking are crucial to provide structural integrity with high cell viability to the printed construct. To address this essential requirement, co-axial extrusion-based bioprinting nozzles have been suggested in the literature and successfully implemented for in vitro and in vivo bioprinting. As shown in FIG. 4A, co-axial nozzle geometry involves two inks extruded from different chambers in a core-shell manner. Literature demonstrates that 3D bioprinting using a co-axial extrusion-based nozzle has better macroscopic and microscopic characteristics than other bioprinting techniques and can protect cells from the external stress and the corresponding damages during printing and crosslinking procedure (e.g., [see Materials (Basel); 2019 (2); 12(4): 640], incorporated herein by reference in its entirety).

For the system, a co-axial nozzle geometry that is compatible with the engineered bioink and the crosslinking procedure was used. Of note, to detail-design a co-axial extrusion-based nozzle 210, various extrusion and printing parameters were considered such as diameters of core and shell chambers (ranging from 250-500 μm), which not only determine the resolution of printing constructs but also affect the printing speed and quality as well as nozzle clogging and flow of bioink during the process. Also, design of the nozzle highly affects and depends on the engineered bioink (e.g., cell size, mechanical properties, and viscosity) and the visible/UV-light-based cross linking procedure. Various extrusion pressures (ranging from 100 to 200 kPa), which determines the deposition flow rate and is affected by diameter of the nozzle tip as well as the diameter and total transmission length from bioink chamber to the nozzle tip, were experimentally explored. Moreover, the viscosity of the engineered bioink affects the precision, uniformity, and speed of bioprinting together with nozzle clogging. Design choices related to bioprinting speed or nozzle translational speed (ranging from 2-10 mm/s) are interconnected to the diameter of the nozzle tip, extrusion pressure, bioink viscosity and deposition flow rate, and pressure loss through the transmission loss. Bioprinting speed also has a direct relationship to nozzle clogging and total printing time. To avoid clogging, a uniform printing speed is required. These interconnected design requirements were considered and studied to optimally design the bioprinting instrument.

In some embodiments, the nozzle 210 may comprise a coaxial nozzle, a single mode nozzle, a multimode nozzle with any suitable number of inputs and outputs, a syringe, and/or a chaotic mixing nozzle (FIGS. 4B-C) which may include a body 250 one or more input channels 251, one or more output channels 252, and one or more mixing stages 253 in a spiral pattern or similar.

Aside from the aforementioned bioprinting requirements, the bioprinting instrument needed to be integrated with a generic redundant robotic manipulator arm 201 (e.g., Kuka LBR, Kuka Inc. or Panda, Franka Emika GmbH) or independently be used as a manual handheld printing instrument, provide an appropriate transmission line and injection control mechanism for a safe, continuous, and uniform injection of the bioink and crosslinking solution from outside to the tip of the co-axial nozzle 210, have a comfortable and ergonomic design to ensure a dexterous and precise micro manipulation by the user, and ensure safety while being co-operated by the surgeon to print the bioink on the surface of the anatomy.

As shown in FIG. 4A, to address these requirements, based on previous experience in developing various medical and surgical instruments and their integration with robotic manipulators (e.g., [see IEEE Trans Med Robot Bionics. 2019 (2), 1(1), incorporated herein by reference in its entirety; and IEEE ASME Trans Mechatron; 2021; 26(1), incorporated herein by reference in its entirety]), a sensorized bioprinting instrument 202 that can be easily integrated and co-operated with a generic robotic manipulator 201 was developed. The instrument has a rigid hollow shaft that provides a safe passage and transmission line for the bioink and crosslinking solution from the external chambers to the co-axial nozzle tip 210. Additionally, a visible/UV light source 207 side-attached to the nozzle tip for facilitating the cross linking procedure was considered (shown in FIG. 4A). The instrument also had an appropriate external bioink storage and injection mechanism.

Also, to precisely and actively control the linear distance of the nozzle tip to the printing surface during procedure, an independent and precise linear DoF with a high-frequency response was considered. This DoF not only provides precise motions in the Z printing direction (i.e., 200-500 μm based on the nozzle diameter) but also its high-frequency response will always ensure a constant distance to the printing surface (e.g., ≤2 mm) through the process to avoid penetrating into the injury site and the printed constructs preventing safety and nozzle clogging concerns. The required constant distance was determined based on the nature of the engineered bioink and other printing parameters. The other required DoFs for manipulation of the instrument were provided by the surgeon and then were filtered and scaled using the robotic manipulator and intelligent control algorithm. To autonomously ensure a constant distance between the nozzle tip and printing surface, a distance measurement sensor 208 (e.g., confocal chromatic miniature distance sensor, Micro-Epsilon, USA) is attached to the nozzle to precisely and continuously measure the distance between the nozzle tip and the surface in a high-frequency rate (shown in FIG. 3A). The measured distance was used in the control algorithm to directly control the independent high-frequency insertion DoF. The instrument also has a small High-Definition camera 209 to provide a high quality real-time video feed of printing procedure that can be displayed in HMD 203.

In some embodiments, the system is configured to print in planar and/or nonplanar (i.e. spatial) trajectories as shown in FIGS. 3B-3C.

### Development of Software and Co-operative Shared Control Components of the SIL-RBP System

The ultimate goal of this task was to integrate the developed bioprinting instrument, robotic manipulator, HMD, 3D point cloud camera, and the optical tracker into a complete SIL-RBP system, shown in FIG. 2A. This required development of novel human-machine interfaces for planning and intuitive HMD augmentation, calibration and registration algorithms, as well as precise and safe semi-autonomous co-operative shared bioprinting control techniques. Here, prior experiences were drawn upon in developing robotic and computer-assisted systems for orthopedic, neurosurgical, laparoscopic, and ophthalmological surgeries (e.g., [see IEEE ASME Trans Mechatron; 2021; 26(3): 1512-1523 incorporated herein by reference in its entirety]; [IEEE ASME Trans Mechatron; 2021; 26(1): 369-380 incorporated herein by reference in its entirety]; and [IEEE Trans Robot. 2020 (2); 36(1): 222-239 incorporated herein by reference in its entirety]). The SIL-RBP software and graphical interfaces were developed based on the “Surgical Assistant Workstation (SAW)” architecture and software libraries developed within the Engineering Research Center for Computer-Integrated Surgical Systems and Technology (see CISST ERC, Johns Hopkins University, incorporated herein by reference in its entirety).

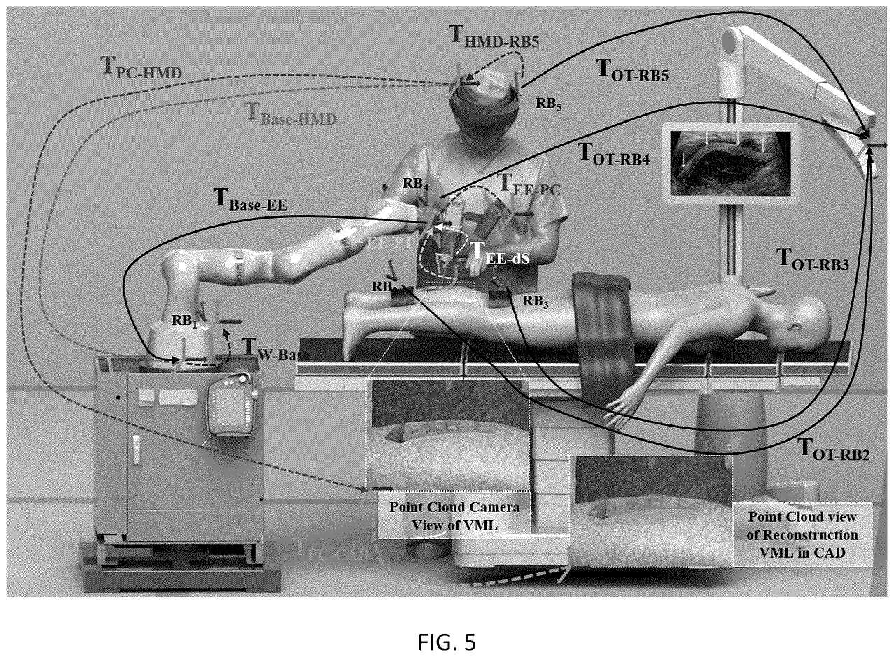

The key step before using the disclosed SIL-RBP system is finding the unknown rigid body transformations (i.e., dashed lines in FIG. 5) between different components of the system using known transformations (shown by solid lines in FIG. 5) obtained by robot 201 kinematics, optical tracker 205, the five rigid bodies 206, and the point cloud camera 204. Here, it is assumed the robotic manipulator 201, optical tracker 205, five optical tracker rigid bodies 206 (labeled by RB₁-RB₅ in FIG. 5), point cloud camera 204, and patient are positioned and fixed appropriately before performing the calibration and registration procedures.

The goal of offline calibrations is to establish constant unknown transformations between different components of the RBP system including the transformations between HMD and optical tracker rigid body RB₅ (T_{HMD-RB5}), world frame and robot base frame (T_{W-Base}), robot end effector frame and point cloud camera frame (T_{EE-PC}). To find these unknown transformations, optical tracker and point cloud camera were used to formulate and apply hand-eye calibration approaches, and the unknown transformations between the robot end effector frame and the bioprinter nozzle tip frame (T_{EE-PC}) and the embedded bioprinter distance measurement sensor (T_{EE-dS}). A pivot calibration technique was used to find these transformations.

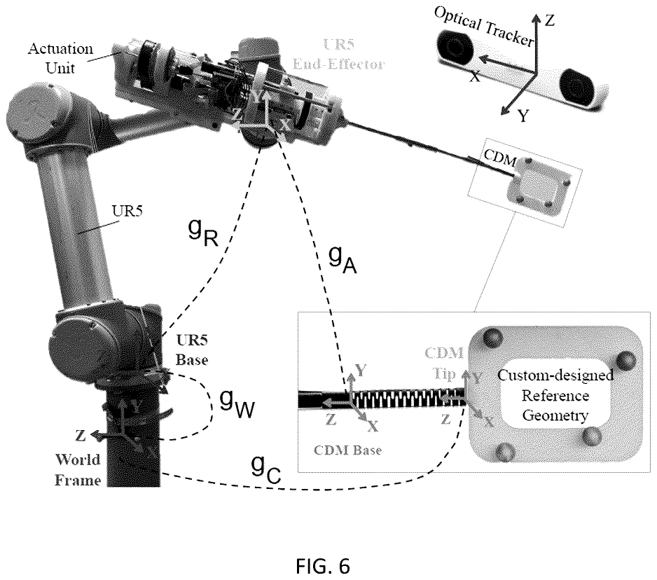

In some embodiments, for example, to register the injury site to the desired reconstructed geometry in the CAD software, the iterative closest point (ICP) algorithm was implemented on the point cloud data of the injury site (obtained with the point cloud camera 204) and the designed 3D geometry of anatomy in CAD software. This transformation has been labeled as T_{PC-CAD} in FIG. 5 and can also be registered dynamically during the printing procedure. The dynamic registration helps to check the discrepancies between the planned printing trajectory and the fabricated constructs to update the planning if necessary and inform the surgeon in real-time using the HMD. This ensures the patient's safety and precision of 3D bioprinting during the closed-loop procedure. To further ensure safety of procedure, inadvertent patient's motions were always monitored using optical tracker and rigid bodies RB₂ and RB₃ placed on two opposite sides of the injury. FIG. 6 shows similar calibration and registration examples implemented by the PI in [see IEEE ASME Trans Mechatron; 2021; 26(1): 369-380, incorporated herein by reference in its entirety; and IEEE Robot Autom Lett.; 2017; (7); 2(3): 1625-1631, incorporated herein by reference in its entirety].

Two control paradigms were developed and implemented to ensure a precise, safe, and intuitive SIL-RBP procedure. For these control algorithms, multi-objective constrained optimization-based control and “virtual fixture” algorithms were adapted (e.g. [see IEEE ASME Trans Mechatron; 2021; 26(1): 369-380, incorporated herein by reference in its entirety, and IEEE Robot Autom Lett.; 2017; (7); 2(3): 1625-1631, incorporated herein by reference in its entirety]).

Semi-Autonomous Co-operative SIL-RBP mode with Visual Augmented Guidance involves the surgeon and computer sharing control of the robotic bioprinting instrument 202 to improve safety and precision of the procedure. More specifically, after performing calibration and registration procedures, the desired printing trajectory, shown by the white color in FIG. 2A, is augmented and displayed in the surgeon's HMD 203 to create a dynamic visual augmented guidance for the user. Next, surgeon holds the bioprinting instrument 202 in hand to follow the displayed desired trajectories while a six-axis force-torque sensor measures in high-frequency and in real-time his/her exerted motion commands. These measurements are used in a high-level admittance control loop to generate scaled and filtered motion velocity commands for the robotic manipulator to accurately manipulate the bioprinting instrument only along the planar desired printing directions (i.e., X_{P}- and Y_{P}-direction shown in FIG. 2A) augmented in the HMD view. A similar control approach has been used on a co-operative retinal surgical robotic system. [see IEEE ASME Trans Mechatron; 2021; 26(3): 1512-1523, incorporated herein by reference in its entirety].

The Z_{P}-direction of bioprinting nozzle 210 is autonomously and precisely controlled using the considered linear DoF and the distance sensor 208 in the bioprinting instrument 202 (shown in FIG. 5). Using this semi-autonomous control mode, the distance between the nozzle tip and surface of anatomy were measured in high frequency during the printing procedure and, if necessary, the described fast-response insertion DoF in the printing instrument automatically adjusts a fixed pre-determined distance (e.g., 2 mm) to compensate the required Z_{P}-direction printing motion. This motion-control-decoupling is essential for the safety and precision (i.e., 200-500±50 μm) of the bioprinting procedure. It is worth emphasizing that through the process, deposition of the bioink and crosslinking is directly controlled by the surgeon and he/she can stop the process if it is necessary.

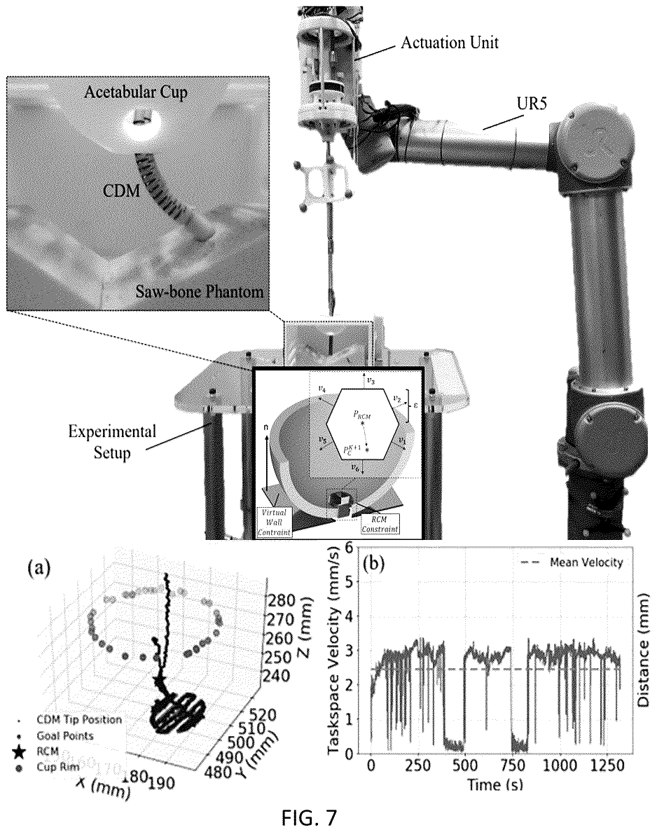

Semi-Autonomous Co-operative SIL-RBP mode with Visual Augmented and Virtual Fixture Guidance includes computer guidance and “virtual fixture” algorithms added to the previous mode. This mode uses simultaneous visual augmented and virtual fixture guidance to modify surgeon's commanding motions in the X_{P}- and Y_{P}-direction of printing to reduce mental processing required to perform this precise task, exceed natural human's precision and performance abilities, reduce the printing time, and create high-quality prints (i.e., 200-500±50 μm). Similarly, Z_{P}-direction control would be autonomously and accurately controlled (i.e., 200-500±50 μm). More specifically, guided virtual fixtures are defined (i.e., two virtual walls shown in FIG. 2A to filter unwanted direction of motion commanded by the surgeon and constrain his/her motions between two virtual walls defined on each side of the desired printing trajectory (i.e., 100 μm apart) to ensure ±50 μm printing resolution in the X_{P}- and Y_{P}-direction, safety, and appropriate printing speed. Of note, the guided virtual fixtures are automatically updated based on the distance measurements, visual feedback, and location of the printing tip with respect to the planned printing trajectory. Moreover, as shown in FIG. 2A, the imposed virtual fixtures were reflected on the HMD Augmented view of printing trajectory. FIG. 7 shows an exemplary application [see IEEE ASME Trans Mechatron; 2021; 26(1):369-380, incorporated herein by reference in its entirety, and IEEE Robot Autom Lett.; 2017; (7); 2(3):1625-1631, incorporated herein by reference in its entirety].

To thoroughly assess the integration of developed hardware and software components of the SIL-RBP system, the performance of the disclosed system was evaluated by in vitro printing of hydrogels/scaffolds and the follow up studies on 3D printed and ex vivo phantom models. These evaluations completely assess the performance of the disclosed RBP system to pave the road for performing in situ animal studies with large VML injuries in the future.

First, N=5 different deformable phantoms were developed simulating various sizes and types of realistic VML injuries-using the J750 Digital Anatomy 3D printer (Stratasys Inc.). TissueMatrix™ and GelMatrix™ materials were used that have been designed and evaluated to mimic native soft tissue behavior. Component-level and system-level evaluations were performed as follows.

The performance of the bioprinting instrument was characterized, assessed, and optimized based on the bioprinting and robotic design criteria listed in herein. More specifically, nozzle clogging, extrusion pressure, deposition flow rate, location, and integration of the distance sensor and light source, together with the linear DoF of the bioprinter and its ergonomic design was thoroughly measured and optimized. Of note, to test the bioprinting parameters, bioink was tested.

In this step, a 7 DoF Kuka LBR robotic manipulator (Kuka Inc.), a Vicon motion capture system (Vicon Motion Systems Ltd., UK) as the optical tracking system, Microsoft HoloLens HMD (Microsoft, Albuquerque, New Mexico, USA), and the Zivid One point cloud camera (Zivid Inc., Norway) were utilized. Using this hardware, the calibration and registration algorithms were implemented and evaluated to find the unknown transformations shown in FIG. 5. A similar procedure has been performed by the PI in IEEE ASME Trans Mechatron; 2021; 26(1):369-380] and [IEEE Robot Autom Lett.; 2017; (7); 2(3): 1625-1631] and shown in FIG. 6.

To evaluate the mentioned SIL-RBP control modes, the system components were integrated to make the system shown in FIG. 2A. Next, the engineered TIBI bioink and the VML phantoms were used to bioprint and reconstruct VML injuries. As listed above, various printing parameters including printing speed (i.e., ranging from 2-10 mm/s), printing resolution in X-, Y-, and Z-direction (i.e., 200-500±50 μm based on the nozzle diameter), and printing time were measured and compared in both control modes to thoroughly assess the system. To assess the bioprinting resolution, appropriate microscopic images were used, and the printing speed and time were measured and recorded. The error was calculated between the planned trajectory and the obtained printing results for both control modes during and after the procedure using the Zivid point cloud camera. Moreover, user studies to collect users' feedback on the printing time and intuitiveness of the control modes were performed. This feedback allows for better development of the HMD visual guidance, virtual fixture, and control algorithm parameters to improve the user performance.

Similar experiments on N=20 ex vivo muscle phantoms of large animals (e.g., lamb and pig) by creating different sizes and types of large VML injuries in them were also performed. These experiments comprehensively evaluated the precision, safety, and performance of the entire system together with the disclosed surgical workflow. For all the performed experiments and trials on phantom and ex vivo models, appropriate statistical analysis was performed such as parametric and non-parametric t-tests, ANOVA to better evaluate and compare the performance of the disclosed control modes. A similar procedure has been previously performed ([see IEEE ASME Trans Mechatron; 2021; 26(1): 369-380, incorporated herein by reference in its entirety, and IEEE Robot Autom Lett.; 2017; (7); 2(3): 1625-1631, incorporated herein by reference in its entirety].

### Quantitative Evaluation of the Print

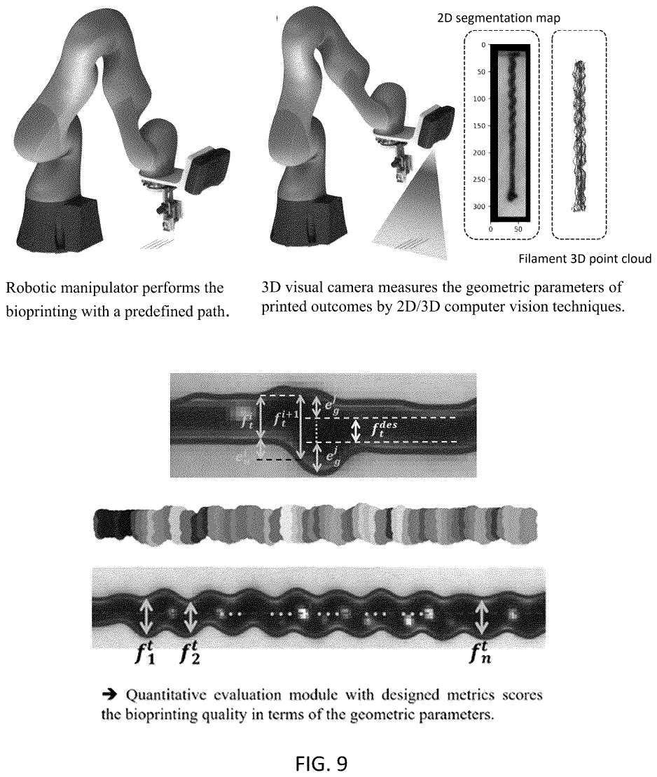

Experiments were performed to quantitatively evaluate the bioprinting process. FIG. 8 shows the experimental robot setup which included a bioprinting tool integrated with a seven-DoF robotic manipulator to precisely perform a generic bioprinting procedure (FIGS. 8-9), a 3D visual measurement system that included a high-accuracy structured light camera with complementary 2D/3D computer vision algorithms to enable online accurate measurement and reconstruction of the geometric parameters of the bioprinted constructs, and a quantitative evaluation module with novel quantitative assessment metrics to quantitatively characterize and evaluate performance of the bioprinting process.

As shown in FIG. 8, the overall components of the robotic system for in situ bioprinting comprised a bioprinting tool, a seven-DoF robotic manipulator (LBR iiwa, KUKA) for precisely controlling the position of the bioprinting tool, and a 3D visual measurement system (Zivid Two M70, Zivid) for quantitatively evaluating and analyzing printing results.

The overall mechanical structure of the experimental bioprinting tool comprised a 50 ml plastic syringe, a linear stepper motor (P850-165-3-ST, Actuonix), a stepper driver (TIC T825, Pololu), a joystick controller, and a microcontroller (Arduino micro, Arduino). The width and maximum length of this system was 58.25 mm and 170.0 mm, respectively.

Considering that the syringe containing the bioprinting gel should be easily replaceable, it was designed to be accessible by removing three rigid parts from the outside. A joystick controller was used to verify normal operation before attaching the proposed system to the robotic manipulator. In addition, considering the properties of bioprinting tools developed in previous studies and the advice of a medical doctor, a linear stepper motor with a maximum force of 25 N, a maximum speed of 15 mm/sec, and a stroke of 50 mm was selected for this study.

Filament thickness plays a fundamental role in scaffold formation, since filament with inappropriate thickness may decrease the pore size of the scaffold or may be prone to becoming disconnected, which results in a degraded biological function. As such, this study focused on measuring printed filament thickness, as shown in FIG. 9.

The overall process for the quantitative characterization of a bioprinting robotic system is summarized in FIG. 9. Specifically, the workflow starts with a preoperative site scanning where the normal map of the pre-printed site (e.g., wound) is acquired via a 3D visual measurement system. Followed by a two-dimensional/three-dimensional (2D/3D) segmentation module, the pixels/sub-pixels belonging to the hydrogels are masked in the color images and the 3D filament point cloud is accessible given the segmented 2D masks (3D segmentation). Next, given the 3D information from both preoperative site scanning and postoperative scanning, the actual sizes of the filaments can be quantified by point cloud processing. To satisfy the strict demand of detecting and measuring the hydrogel filament, the system is mainly based on the 3D industrial camera due to its high resolution (2.3 Mpix), precision point clouds (<60 μm), and the capability of detecting reflective and shiny objects, which allows the acquisition of the colored images and point cloud information with high quality.

To assist the following thickness calculation, a normal map of the printing site is preoperatively captured by using the 3D camera in the preoperative site scanning stage. Note that this scanning is only performed at the beginning of the workflow. In each postoperative scanning, the acquired normal map serves to determine a projection surface that is parallel with the thickness dimension.

Given the colored images from postoperative scanning with printed gels, a 2D/3D segmentation is performed to mask filament pixels and 3D points, respectively. The challenges of segmenting the gel filaments can be attributed to two aspects: (1) the small scale of filament thickness makes the measurement highly error-prone since one incorrectly segmented pixel can lead to a measurement error of at least 0.18 mm; (2) the minimum working distance of the structured-light camera degrades the image resolution and thus limits the acquisition of the fine details of the filament boundaries.

To this end, the GrabCut algorithm (see C. Rother et al., ““GrabCut” interactive foreground extraction using iterated graph cuts,” ACM transactions on graphics (TOG), vol. 23, no. 3, pp. 309-314, 2004; incorporated herein by reference in its entirety) was used as the backbone to develop an interactive segmentation framework that only needs sparse scribbles as annotations. This algorithm uses texture (color) information and boundary (contrast) information of the image, which minimizes the amount of user interaction to obtain better segmentation performance. With the segmentation map attained, an interactive correction is allowed to correct those misclassified regions. Due to the increase in manual intervention, it is more accurate than automatic segmentation. These two procedures are conducted iteratively until the segmentation map achieves gold standard-level segmentation accuracy, which means no obvious errors can be visually found in images. Typically, this loop only needs to repeat about 1 to 2 times for each image, which matches the need for time efficiency.

Although pixel-level perfect segmentation has been achieved via the developed interactive segmentation algorithm, aliasing still exists in the boundary regions due to the resolution limitation, which can impair the point cloud measurement to some extent. Therefore, a region-based level set method is leveraged to refine the segmentation predictions by using the mask boundary from the interactive segmentation stage as the initial contour and actively adjusting the contour to approach the filament boundary (see C. Li et al., “Distance regularized level set evolution and its application to image segmentation,” IEEE transactions on image processing, vol. 19, no. 12, pp. 3243-3254, 2010; incorporated herein by reference in its entirety). The contour can implicitly evolve toward the filament boundary by minimizing the energy function as follows:

$$E ( {c}_{1} , {c}_{2} , Φ ) = μ \cdot Length ( Φ ) + ν \cdot Area ( Φ ) + {λ}_{1} {\int }_{Ω} {\vert {μ}_{0} ( x , y ) - {c}_{1} \vert}^{2} H ( Φ ( x , y ) ) dxdy + {λ}_{2} {\int }_{Ω} {\vert {μ}_{0} ( x , y ) - {c}_{2} \vert}^{2} ( 1 - H ( Φ ( x , y ) ) ) dxdy \qquad ( 1 )$$

where φ denotes the contour function, (x,y)∈Ω denotes the location within the domain of image Ω, c₁, c₂ respectively represent the mean intensity values of the interior and exterior of the contour regions, μ, v, λ₁ and λ₂ are fixed parameters. Length(φ) and Area(φ) represent the length and inside area of the contour, respectively, which serve as regularization. H is the Heaviside Function (HF):

$$H ( s ) = \{ \begin{matrix}1 , & s \geq 0 \\ 0 , & s < 0\end{matrix} \qquad ( 2 )$$

With the nature of the continuous function, the level set function can generate a sub-pixel-level continuous boundary for the segmentation mask and further mitigates the errors from resolution. Besides, considering that the initial segmentation mask has reached pixel-level accuracy, the regularization terms Length(φ) and Area(φ) can prevent complex and unrealistic boundaries during the contour evolution.

Given the sub-pixel-level segmentation map, the algorithm masks the points belonging to the hydrogel filaments for postoperative scanning. Considering the dense distribution of the point clouds near the boundary regions, bilinear interpolation can be adopted to estimate the depth values of the sub-pixel contour points.

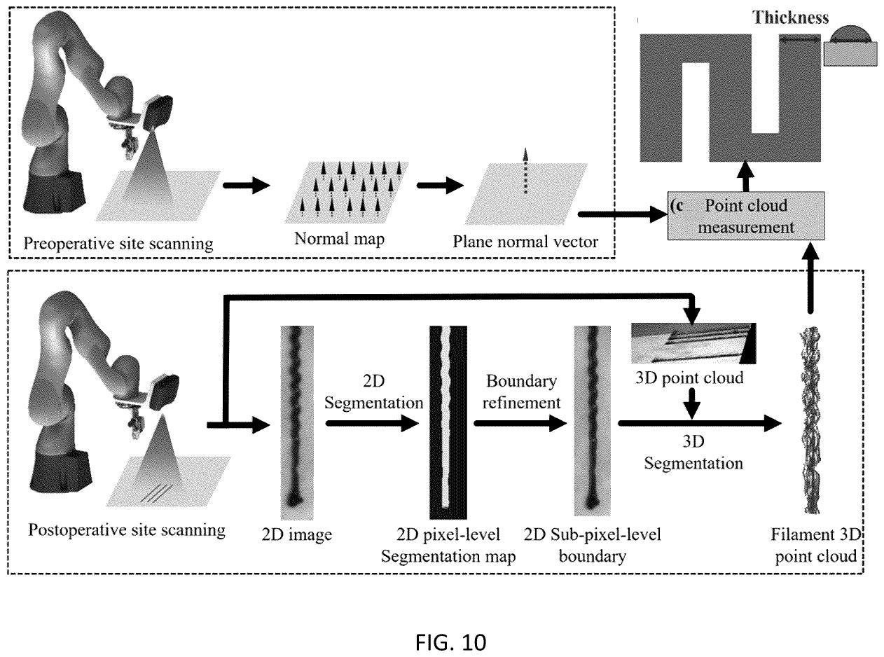

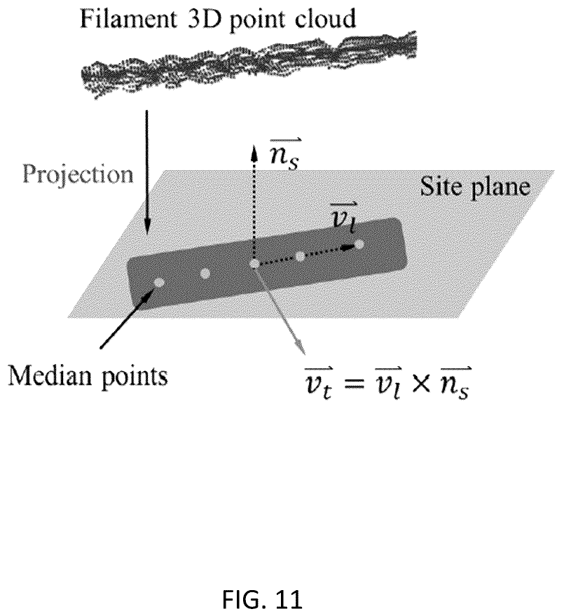

Point cloud measurement (FIGS. 10-11) served to obtain the thickness measurement given the normal vectors from preoperative site scanning and the filament 3D point cloud. To simplify the thickness calculation, the point cloud of printed filaments was projected to the preoperative site plane along the corresponding normal vector.

Furthermore, a skeleton extraction is performed to extract all the median points from the projected point clouds. With a large number of median points, the printed gel strand can be split into a sequence of elements determined by two neighboring median points. Also the filament direction [symbol] in FIG. 11 can be fit given the median points. Then for each element, by calculating the cross-product of plane normal vector [symbol] and filament direction [symbol] , the thickness dimension [symbol] where thickness values

$${f}_{t}^{i}$$

of the i-th element can be calculated by subtracting the maximum value and the minimum value along the thickness direction [symbol] .

To fully exploit the quantified thickness, two new metrics were defined representing the global error e_{g} and a uniformity error eᵤ for evaluating the performance of each segment in the filament, which is defined as follows:

$${e}_{g}^{i} = ‖ {f}_{t}^{i} - {f}_{t}^{des} ‖ \qquad ( 3 )$$

$${e}_{u}^{i} = ‖ {f}_{t}^{i} - {f}_{t}^{i - 1} ‖ \qquad ( 4 )$$

where the

$${e}_{g}^{i} and {e}_{r}^{i}$$

represent the Global Error and Uniformity Error of i-th element, respectively,

$${f}_{t}^{i}$$

denotes the thickness value of the i-th filament element, the

$${f}_{t}^{des}$$

denotes the desired thickness value of the measured filament which can be determined by the needle inner diameter and the identified sensor errors. The global error e_{g} reflects the accuracy of the printing outcomes in the i-th element and the uniformity error eᵤ indicates the uniformity based on two neighboring elements. These two metrics are applied in element-wise quantitative evaluation for printed filaments. The global error serves to quantify the accuracy of the printed filaments. In addition, the Overall Global Error E_{g} and Overall Uniformity Error Eᵤ are defined as follows:

$${E}_{g} = \frac{{\sum }_{1} {e}_{g}^{i}}{N} \qquad ( 5 )$$

$${E}_{u} = \frac{{\sum }_{1} {e}_{u}^{i}}{N} \qquad ( 6 )$$

where the N denotes the total number of elements in the measured filament. With these metrics, one can access both the continuous and the filament-wise quantitative evaluations, which further support the parameter characterization of the robot system for reaching the desired performance.

Since most of the experimental analysis and evaluation in this study were performed through a 3D visual measurement system, it was necessary to verify the measurement accuracy of this system. Two experiments were performed to verify the accuracy of the 3D visual measurement system and characterize the parameters for the robotic system which minimizes E_{g} and Eᵤ defined in Eq. (5) and Eq. (6), respectively.

An overview of the experimental environment is shown in FIG. 8. To evaluate the effectiveness and accuracy of the 3D visual measurement system, a customized calibration board was designed where grooves with pre-designed widths of 3 mm, 2 mm, and 1 mm were engraved. A blue-dyed ultrasound gel (Aquasonic) was adopted to simulate the hydrogel considering similar characteristics.

During the experimental process, the scan of the calibration board filled with gels was processed by the developed visual measurement system to calculate the filament thickness. With the known ground-truth width of the designed grooves, the errors defined in Eq. (5), (6) were calculated with the desired filament thickness

$${f}_{t}^{des}$$

set as the corresponding groove width. Additionally, for each ground-truth thickness value, an ablation study was also performed for the sub-pixel-level refinement (SPR). ‘w/SPR’ means the level set method was used to perform a sub-pixel level refinement, and ‘w/o SPR’ means only the pixel-level segmentation map was used. For the quantitative results, all experiments were repeated three times and the average was found.

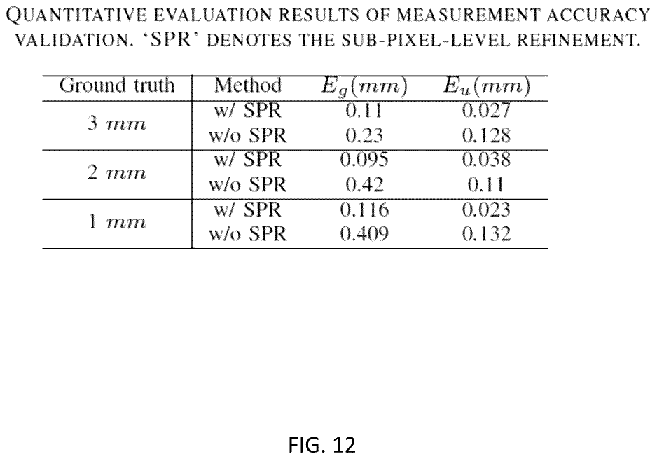

The quantitative results are presented in the table of FIG. 12, which shows the overall global error E_{g} and global uniformity error Eᵤ for gel strands with a ground truth thickness of 3 mm, 2 mm, and 1 mm, respectively. Note that for each experiment shown in the table, the global error was calculated by setting the desired thickness

$${f}_{t}^{i}$$

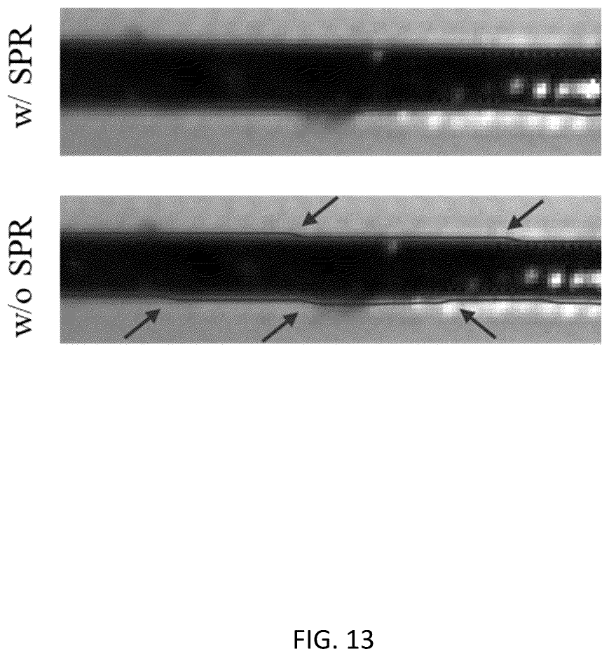

as the corresponding ground-truth thickness values. It can be observed that the sub-pixel-level refinement (SPR) reduced the errors considerably and led to stable global errors with various ground truth thicknesses. On the contrary, ‘w/o SPR’, which means only using pixel-level segmentation maps is erroneous. The comparison between ‘w/SPR’ and ‘w/o SPR’ are visualized in FIG. 13, in which the smoothed boundary can be witnessed after the refinement. Considering that the needle used in the following experiment has an inner diameter of 0.838 mm, which is the minimum thickness the robotic system can ideally reach, this result validated the performance of the system. Therefore, the measurement error Eₑₛₑₙₛₒᵣ is approximately estimated by using E_{g} given the closest ground truth of 1 mm.

The main goal of this experiment was to find parameters related to robot-assisted bioprinting that can minimize E_{g} and Eᵤ defined in Eq. (5) and Eq. (6), respectively. Considering the overall concept of robot-assisted bioprinting, the robot velocity vᵣ (unit: mm/s), extrusion rate rₑ (unit: μL/s), and the distance between the needle tip and surface Dₜᵢₚ (unit: mm) were selected as main characterization parameters that can mainly affect the printing performance. An 18-gauge needle, which is most commonly used for bioprinting, was used in this experiment, and the inner diameter of this needle d_{needle} was about 0.838 mm.

Although the thickness of the printed filament should ideally be equal to the d_{needle}, considering the sensor error of the 3D visual measurement system \tilde{E}ₛₑₙₛₒᵣ that measure the thickness, the desired thickness

$${f}_{t}^{d e s}$$

as defined as follows:

$${f}_{t}^{des} = {d}_{n e e d l e} + {\tilde{E}}_{s e n s o r} \qquad ( 7 )$$

Here, since the d_{needle} is close to 1 mm, the E_{g} of gel strand with the ground-truth thickness of 1 mm was used for Eₛₑₙₛₒᵣ. Therefore,

$${f}_{t}^{des}$$

was set to 0.954 mm in this case.

To find the optimal combination of the parameters, a case-wise characterization was performed by conducting a grid search experiment where the search space is vᵣ∈{1, 2, 3, 4, 5, 6} mm, rₑ∈{4, 8, 12} μm/L, Dₜᵢₚ∈{0.8, 1.8, 2.8} mm. Thereby one can find the combination leading to the best performance (i.e., minimum E_{g} and Eᵤ). For simplicity, the index

$${Case}_{{v}_{r} , {r}_{e}}^{{D}_{tip}}$$

was used to represent one certain case where the robotic system performed with corresponding vᵣ, rₑ and Dₜᵢₚ. Next, to verify the effectiveness of the developed quantitative characterization system, several cases were selected to quantify and visualize their continuous errors with designed metrics.

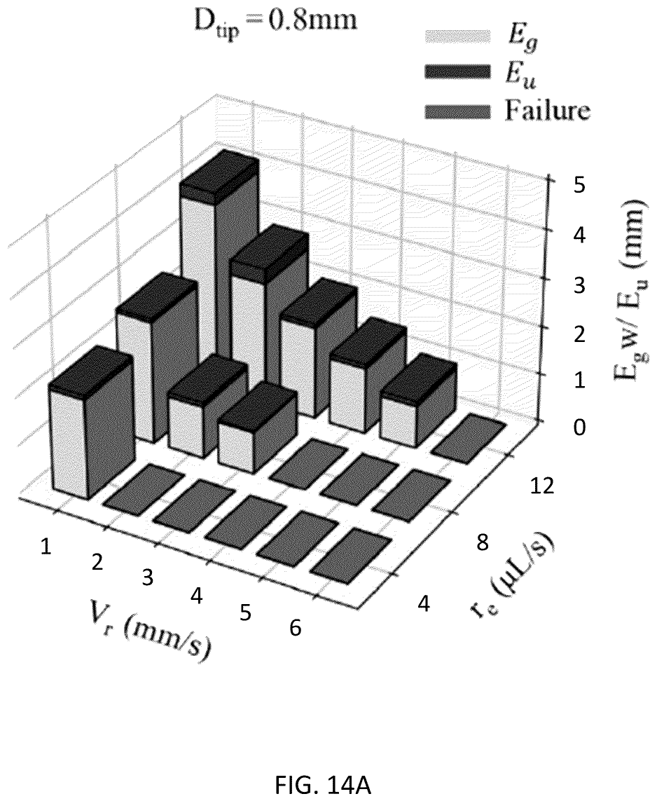

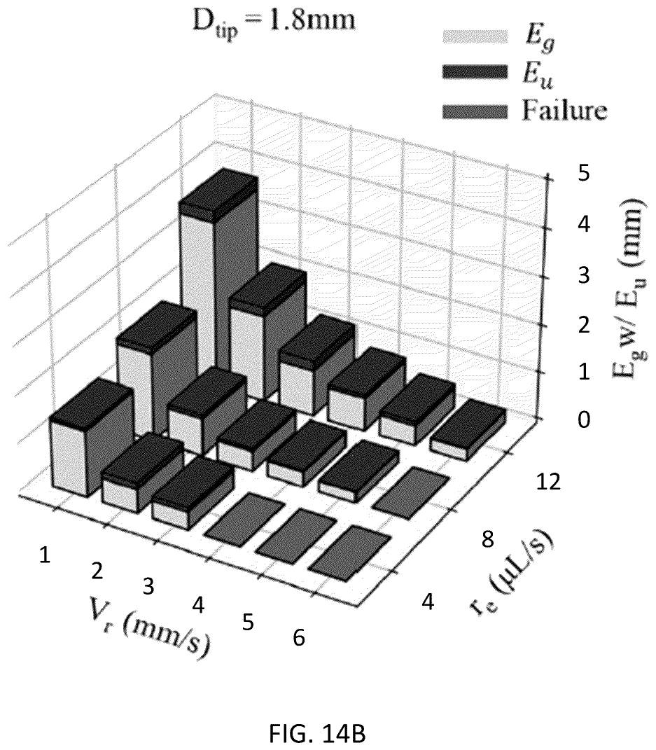

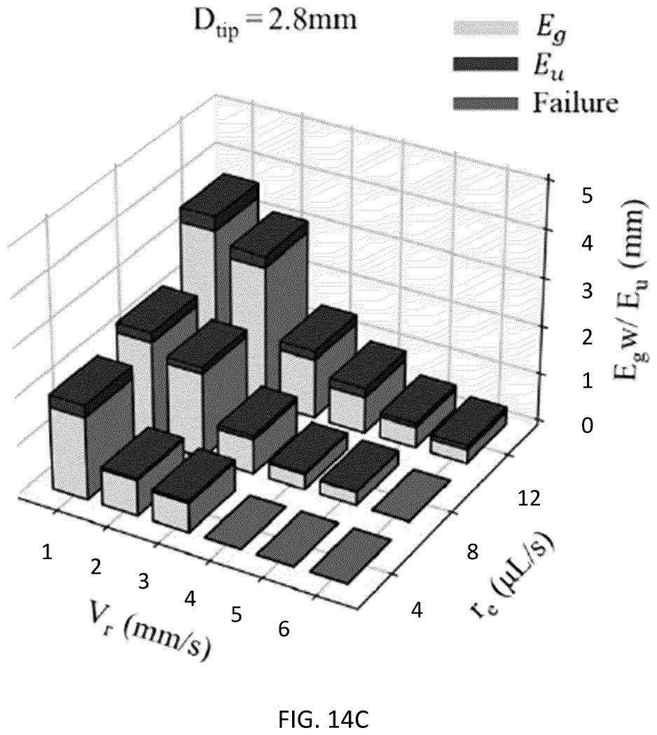

The case-wise characterization results are shown in FIGS. 14A-14C, where the overall global error E_{g} and overall uniformity error Eᵤ are calculated with different vᵣ, rₑ, and Dₜᵢₚ. In this bar chart, small values of E_{g} and Eᵤ respectively represent the high accuracy and high uniformity of the printed gels. The robotic system reached the optimal performance at Case_{5,8}^{1.8}, where the E_{g}=0.23 mm and Eᵤ=0.01 mm. It can be observed from FIG. 14A that, small Dₜᵢₚ can increase the risk of discontinuous filaments. Cases with Dₜᵢₚ=1.8 mm (FIG. 14B) and Dₜᵢₚ=2.8 mm (FIG. 14C) shared similar trends where both of them reached minimum errors at the combination of vᵣ=5 mm and rₑ=8 L/s and detected failure cases by increasing the vᵣ from 5 mm.

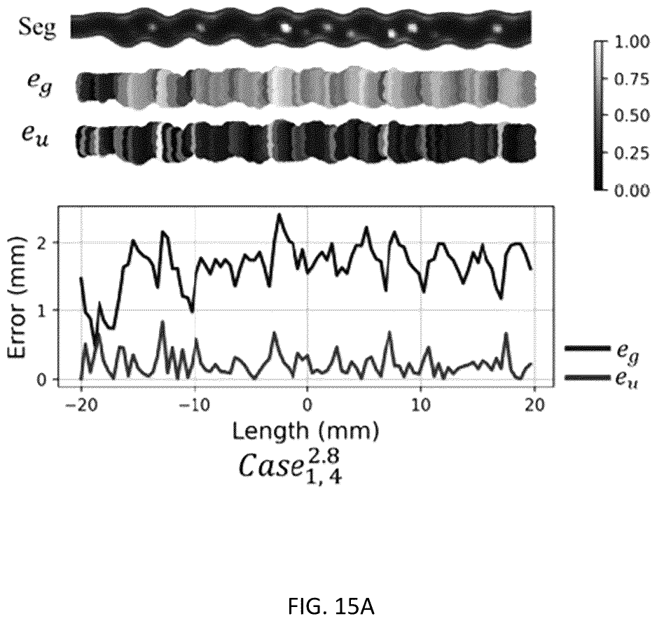

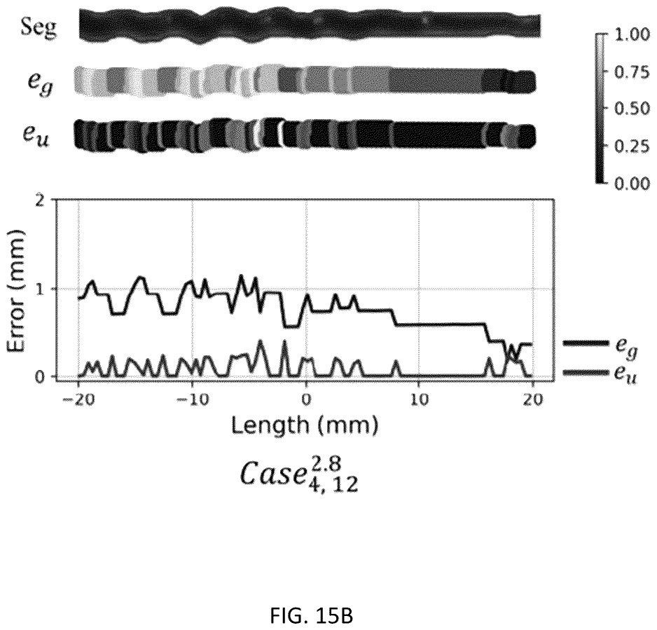

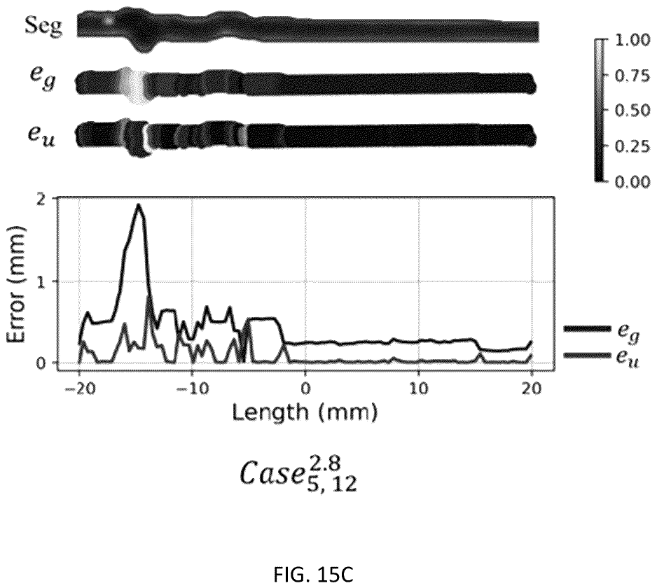

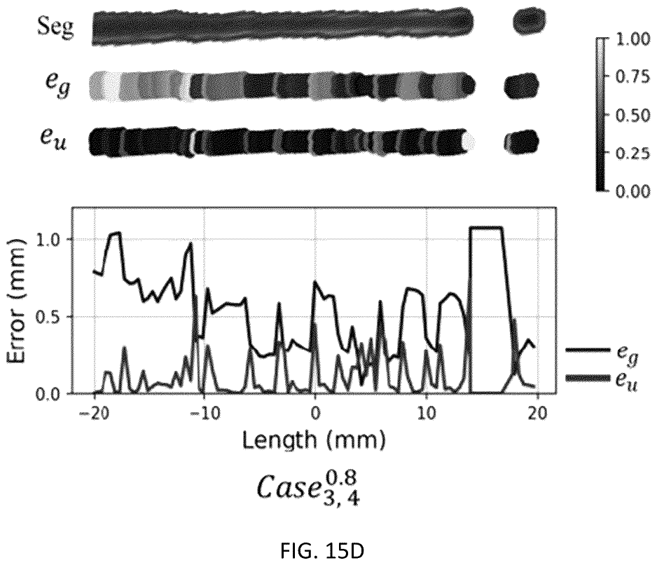

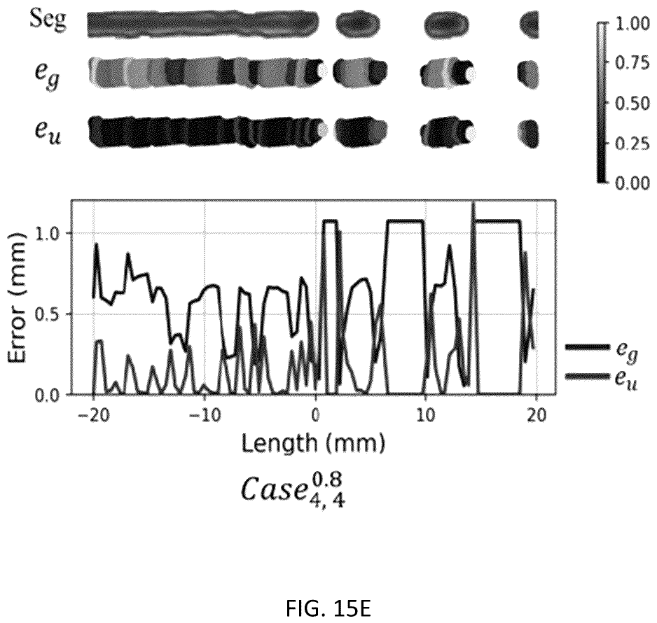

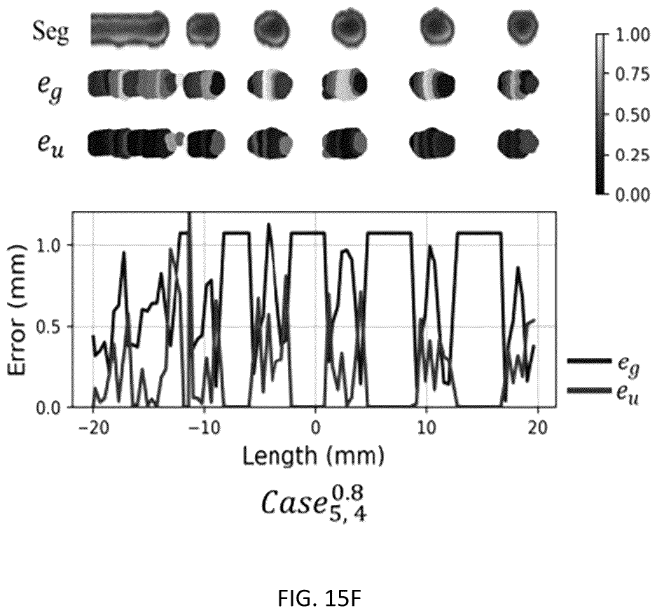

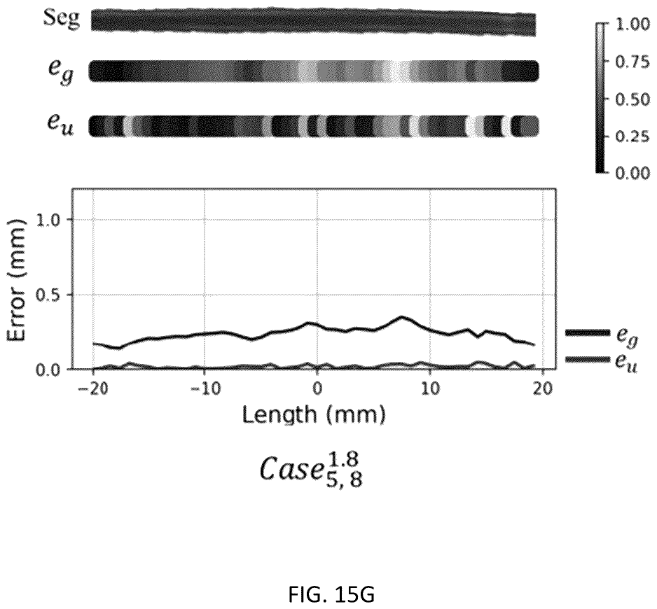

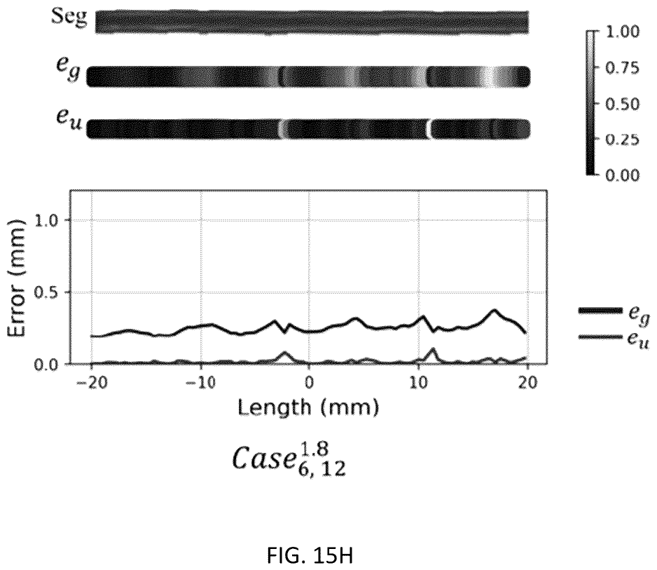

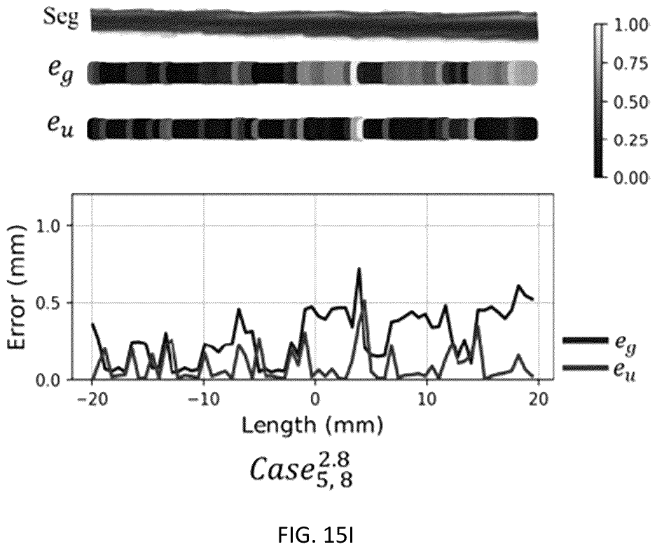

FIGS. 15A-15l present some continuous quantitative evaluation on some informative cases in which the normalized continuous global errors and uniformity errors are visualized as heat maps for the whole filament and aligned with the segmented colored images. The measured errors for each case (without considering the failure cases): (a) E_{g}=1.65 mm, Eᵤ=0.21 mm (b) E_{g}=0.73 mm, Eᵤ=0.07 mm (c) E_{g}=0.41 mm, Eᵤ=0.08 mm (g) E_{g}=0.23 mm, Eᵤ=0.01 mm; (h) E_{g}=0.26 mm, Eᵤ=0.01 mm; (i) E_{g}=0.28 mm, Eᵤ=0.18 mm.

FIGS. 15A-15C shows the continuous errors for cases from which large global errors and low uniformity can be observed. FIGS. 15D-15F shows the failure cases with disconnected gel filaments. FIGS. 15G-15I visualize several well-performing cases which indicate similar overall global errors in the case-wise characterization. The overall global errors for FIGS. 15G-15I are 0.23 mm, 0.28 mm, and 0.26 mm, respectively, which is hard to visually compare.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention.

1. A bioprinting instrument, comprising:
a housing including first and second ends;
a distance measurement sensor positioned on the housing proximate to the first end;
a light source positioned on the housing proximate to the first end;
a 3D point cloud camera positioned on the housing; and
a nozzle positioned on a one degree of freedom (DoF) linear height control mechanism positioned on the housing proximate to the first end.

2. The bioprinting instrument of claim 1, wherein the bioprinting instrument is configured to be held by a surgeon or is configured for in situ deposition of bioink.

3. (canceled)

4. The bioprinting instrument of claim 1, wherein the bioprinting instrument is configured for in situ deposition of bioink the bioink comprising at least one of gelatin-methacryloyl (GelMA) incorporating sustained insulin-like growth factor-1 (IGF-1) and myoblast cells, alginate, gelatin, collagen, fibrin, fibrinogen, gellan gum, silk, hyaluronic acid, dextran, agarose, chitosan, hydroxyapatite, a decellularized matrix based bioink, a growth factor based bioink, Matrigel, PCL, PEG, Pluronic, HAMA-pHPMA-lac/PEG, PG-HA, PVP, a cell aggregate bioink, a pellet based bioink, Derma-matrix, Novogel, AuNPs, AgNPs, magnetic iron oxide particles, blood plasma, a cryo bioink, ultrashort peptides, a genetically engineered phage, and a conductive bioink.

5. The bioprinting instrument of claim 1, wherein the nozzle has a diameter of 50 μm to 1000 μm and comprises a coaxial nozzle, a single mode nozzle, a multimode nozzle, a syringe, or a chaotic mixing nozzle.

6. The bioprinting instrument of claim 1, wherein the light source comprises a visible/UV light source.

7. The bioprinting instrument of claim 1, wherein the bioprinting instrument utilizes an extrusion pressure of 0.1 kPa to 500 kPa.

8. A bioprinting robotic system, comprising:
a robotic manipulator;
the bioprinting instrument of claim 1 connected to and configured as an end effector of the robotic manipulator, further configured to print bioink;
a head mounted display (HMD);
an optical tracking system; and
a computing system communicatively connected to the robotic manipulator, bioprinting instrument, HMD and optical tracking system.

9. The system of claim 8, wherein the computing system comprises:
a processor and a non-transitory computer-readable medium with instructions stored thereon, which when executed by a processor, perform steps comprising:
obtaining a high-resolution geometry of a volumetric muscle loss (VML) injury via the 3D point cloud camera;
designing a desired 3D printing geometry and corresponding printing trajectories;
calibrating and registering the bioprinting robotic system; and
displaying on the HMD the desired printing trajectory.

10. The system of claim 8, wherein the robotic manipulator comprises a 6 or 7 degrees of freedom redundant robotic manipulator.

11. The system of claim 8, further comprising rigid body markers utilized by the optical tracking system as reference points for calibration, registration, and real time tracking of the system components and the patient.

12. The system of claim 8, wherein the system is configured to operate in
a) a semi-autonomous cooperative SIL-RBP mode with visual augmented guidance;
b) in a semi-autonomous cooperative SIL-RBP mode with visual augmented guidance virtual fixture guidance mode
c) an assistive mode in which a surgeon holds the robot or bioprinting instrument and the system corrects the motions of the surgeon;
d) a semi-autonomous mode in which a portion of the motions are controlled by a surgeon and the remaining portion of the motions is controlled by the robot;
e) a semi-autonomous mode in which a portion of the motions are controlled by a surgeon and the remaining portion of the motions is controlled by the robot, the robot being configured to correct the motions of the surgeon;
f) a tele bioprinting mode in which a surgeon remotely controls a remote robot via another user interface or a leader robotic system;
g) a tele bioprinting mode in which a surgeon remotely controls a remote robot via another user interface or a leader robotic system, the robot being configured to correct the motions of the surgeon;
h) an autonomous mode in which the entirety of the bioprinting can be controlled autonomously based on the determined printing trajectory.

13. (canceled)

14. The system of claim 8, wherein the robotic manipulator, the bioprinting instrument, the HMD, the optical tracking system and the computing system are communicatively connected via wired, wireless, or wired and wireless means.

15. The system of claim 8, wherein the system utilizes co-operative shared control and virtual fixture algorithms to ensure safety and precision during the bioprinting procedure.

16-20. (canceled)

21. The system of claim 8, wherein the system is configured to operate in a tele bioprinting mode in which a surgeon remotely controls a remote robot via another user interface or a leader robotic system, and wherein the system is configured to provide haptic, audio or visual feedback to the surgeon.

22. (canceled)

23. The system of claim 8, wherein the system is configured to perform spatial or planar bioprinting.

24. A bioprinting method, comprising:
providing the bioprinting robotic system of claim 8;
obtaining a high-resolution volumetric geometry of an injury via the 3D point cloud camera;
designing a desired 2D or 3D printing geometry and corresponding printing trajectories;
calibrating and registering the bioprinting robotic system;
displaying on the HMD the desired printing trajectory; and
accepting input from a surgeon to print along the desired printing trajectory via a manipulation of the bioprinting instrument.

25. The method of claim 24, further comprising
a) utilizing an algorithm to guide and scale the surgeon's movement to provide precise micromanipulation; and/or
b) monitoring the procedure and inadvertent patient movement via the optical tracker and 3D point cloud camera to cancel or correct the surgeon's manipulation of the bioprinting instrument.

26. (canceled)

27. The method of claim 24, wherein the step of designing a desired 3D printing geometry and corresponding printing trajectories comprises considering the point cloud data, clinician's feedback, properties of the engineered bioink, and biomechanics of the anatomy.

28. The method of claim 24, wherein the step of calibrating and registering the bioprinting robotic system comprises utilizing the optical tracking system and software to calibrate and register the printing instrument, HMD, 3D point cloud camera, and the robotic manipulator.

29. A bioprinting robotic system, comprising:
a robotic manipulator;
a bioprinting instrument connected to and configured as an end effector of the robotic manipulator, further configured to print bioink;
a display;
an optical tracking system; and
a computing system communicatively connected to the robotic manipulator, bioprinting instrument, display and optical tracking system, comprising a processor and a non-transitory computer-readable medium with instructions stored thereon, which when executed by a processor, perform steps comprising:
obtaining a high-resolution geometry of a target site via a 3D point cloud camera;
designing a desired 3D printing geometry and corresponding printing trajectories;
calibrating and registering the bioprinting robotic system; and
displaying on the desired printing trajectory.

30-31. (canceled)
